(19) United States
(12) Patent Application Publication
(10) Pub. No.: US 2009/0253982 A1
(43) Pub. Date: Oct. 8, 2009

(54) ASSESSING SUBJECT'S REACTIVITY TO PSYCHOLOGICAL STRESS USING FMRI

(76) Inventor: Jiongjiong Wang, Cherry Hill, NJ (US)

Correspondence Address: Pearl Cohen Zedek Latzer, LLP, 1500 Broadway, 12th Floor, New York, NY, 10036, US

(21) Appl. No.: 12/295,852

(22) PCT Filed: Apr. 3, 2007
(86) PCT No.: PCT/US07/08065

§ 371 Date: Mar. 17, 2009

Related U.S. Application Data
(60) Provisional application No. 60/788,120, filed on Apr. 3, 2006.

(51) Int. Cl. A61B 5/055

(52) U.S. Cl. 600/419

(57) ABSTRACT

This invention relates to the use of a quantitative functional MRI (fMRI)—arterial spin-labeling perfusion MRI or absolute T2 mapping MRI or a combination thereof in the non-invasive neuroimaging of a subject's brain in response to stress-inducing psychological stimuli, which can be utilized to predict individual stress reactivity as well as to be used as a human model for testing or optimizing psychopharmacological agents.

### FIELD OF INVENTION

This invention is directed to a method for identifying individuals with resilience or susceptibility to psychological stress. Specifically, the invention relates to the use of a quantitative functional MRI (fMRI)—arterial spin-labeling perfusion MRI or absolute T2 mapping MRI or a combination thereof in the non-invasive neuroimaging of a subject's brain in response to stress-inducing psychological stimuli and as a human model for testing psychopharmacological agents.

### BACKGROUND OF THE INVENTION

Stress is common in everyday life and is believed to affect happiness, health, and cognition. Stress reactivity and susceptibility are important elements in screening candidates for high stress tasks, including top executives, elite athletes, astronauts, air traffic controllers and combat soldiers etc. Stress reactivity is also important in identifying risk populations for developing stress/anxiety related disorders such as depression, phobia, post-traumatic stress disorder, insomnia, drug addiction and vulnerability to infection etc. To date, there are no exclusive testing procedures to determine if an individual is resilient or susceptible to negative effects of stress. Neurocognitive assessments (questionnaire) are often used to profile personality, however, the causal relationship between particular personality dimensions/factors and stress reactivity remains elusive. Additionally, there remains the possibility that candidates may conceal their character through conscious deception or malingering. Physiological stress assessments including measuring activity of the sympathetic nervous system (e.g., heart rate and blood pressure), and assay of hormones related to the hypothalamus-pituitary-adrenal (HPA) axis (e.g., serum and salivary cortisol). However, these parameters indicate peripheral responses that are delayed in time and generally reflect the integrated activity of several biological systems. It will be highly preferable to directly visualize the stress effect in the human brain.

Reliable biomarkers of stress reactivity are also needed for developing, optimizing and testing pharmacological interventions for the prevention and treatment of stress related disorders. To date, valid models for human psychiatric diseases are very limited. Testing candidate psychopharmacological agents during pre-clinical and clinical human trials require considerable sample size. Time and cost in order to observe significant results in terms of behavioral symptoms.

During recent years, although considerable progress has been made in uncovering the neuroendocrine and molecular processes mediating the cascade of reactions to stressors, the central mechanism and neural correlates of psychological stress in human brain remain unknown. Hence, a reliable central marker of the stress effect is lacking. Manifestations of the fight-or-flight response under life-threatening situations suggest that the brain's response to stress may (at least) involve excitation of the emotion and vigilance systems and inhibition of appetitive goals. For instance, a prey evading a predator is in constant fear and high alert, with suppressed function for food intake and reproduction. Although the majority of stress today is due to psychosocial factors and is not life-threatening, this stereotyped brain-activation pattern may still take place during a test, a job interview or an impromptu speech. This hypothesis is supported by neurochemical studies indicating that a common denominator of the response to stress in the brain, secretion of corticotrophin-releasing hormone and norepinephrine, causes symptoms including arousal, fear-related behavior, and suppressed appetite. These characteristic neural activation patterns under stress may be captured using modern neuroimaging techniques, which in turn can be utilized to predict individual vulnerability to psychosocial stress as well as to evaluate treatment of stress related disorders.

Recent neuroimaging studies have enriched understanding of the neuroanatomical substrates underlying perception, cognition, and emotion. Data on emotional processes suggest a common neural network involving the prefrontal cortex, amygdala, insula, basal ganglia, and anterior cingulate. In particular, negative affect generally elicits activation in the right prefrontal cortex (RPFC), amygdala, and insula, whereas the left prefrontal cortex is associated with positive emotion and appetitive goals along with reward-related cortical regions. The neural correlates of vigilance and sustained attention have been largely localized to the right prefrontal and parietal lobe and the thalamus. However, little direct neuroimaging evidence is available concerning the central mechanism of the stress response. To date, only a few PET (positron emission tomography) studies attempted to measure cerebral blood flow changes during stress tasks. The use of PET is limited by the repeated exposure to radioactivity during a single scanning session. Although BOLD (blood oxygenation level dependent) fMRI is the mostly widely used neuroimaging method, the use of BOLD fMRI for studying the central effect of psychological stress is limited by the intrinsic baseline drifts in BOLD signal, rendering poor sensitivity in visualizing sustained behavioral states such as stress.

### SUMMARY OF THE INVENTION

In one embodiment, the invention provides a method for differentiating a subject's reactivity to psychological stress comprising: establishing a cerebral blood flow (CBF) perfusion baseline, blood oxygenation baseline, or their combination for the subject, using scanning with arterial spin labeling (ASL) perfusion magnetic resonance imaging (MRI) or absolute T2 mapping MRI; inducing stress in the subject, while the subject is undergoing MRI scanning with arterial spin labeling (ASL) perfusion magnetic resonance imaging (MRI) or absolute T2 mapping MRI; capturing changes in the cerebral blood flow (CBF), blood oxygenation, or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow, blood oxygenation pattern or their combination with changes in blood flow, blood oxygenation pattern or their combination in a reference database, wherein the reference database indicates reactivity to psychological stress of a predetermined individual or pool of individuals.

In another embodiment, the invention provides a method of screening candidates for a high-stress position comprising the steps of: establishing a cerebral blood flow (CBF) perfusion baseline, blood oxygenation baseline or their combination for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF) perfusion, blood oxygenation or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern, or their combination with changes in blood flow pattern, blood oxygenation pattern, or their combination in a reference database, wherein the reference database indicates reactivity to psychological stress of a predetermined individual or pool of individuals proven as appropriate for the high-stress position sought to be screened for.

In one embodiment, the invention provides a method of diagnosing a mental disorder associated with a subject's susceptibility to psychological stress comprising the steps of establishing a cerebral blood flow (CBF) perfusion baseline, blood oxygenation baseline or their combination for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF), blood oxygenation, or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern, or their combination with changes in blood flow pattern, blood oxygenation pattern, or their combination in a reference database, wherein the reference database indicates reactivity to psychological stress of a predetermined individual or pool of individuals correctly diagnosed with said mental disorder sought to be diagnosed.

In another embodiment, the invention provides a library of images of cerebral blood flow changes, blood oxygenation changes or their combination in brain regions associated with stress response, wherein the images are captured in response to psychological stress, using scanning with arterial spin labeling (ASL) perfusion magnetic resonance imaging (MRI) or absolute T2 mapping MRI, taken from a predetermined subject or pool of subjects.

In one embodiment, the invention provides a method of testing a candidate drug as an psychotherapeutic drug, comprising the step of: deviding a cohort of subjects into two groups, administering to one group a placebo and to the other group the candidate drug; establishing a cerebral blood flow (CBF) perfusion baseline, blood oxygenation baseline or their combination for both groups, using scanning with arterial spin labeling (ASL) perfusion magnetic resonance imaging (MRI) or absolute T2 mapping MRI; inducing stress in both groups, while individuals in the groups are undergoing scanning with arterial spin labeling (ASL) perfusion magnetic resonance imaging (MRI) or absolute T2 mapping MRI; capturing changes in the cerebral blood flow (CBF), blood oxygenation or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern, or their combination between the individuals in the group that received placebo, with the individuals in the group that received the candidate drug, wherein blood flow pattern, blood oxygenation pattern, or a combination thereof in the group which received the candidate drug, which yields cerebral blood flow pattern, blood oxygenation pattern, or a combination thereof, which resembles the baseline cerebral blood flow pattern, blood oxygenation pattern, or a combination thereof, which is closer than that of the cerebral blood flow pattern, blood oxygenation pattern of a combination thereof, of the group that received placebo, indicate the candidate drug is an psychotherapeutic drug.

In another embodiment, the invention provides a method of optimizing a psychopharmacological agent for a psychiatric condition, comprising the steps of: dividing a cohort of subject exhibiting the psychiatric condition for which the psychopharmacological agents are sought to be optimized, to a number of groups equal to the number of psychopharmacological agents sought to be optimized; administering the psychopharmacological agents to the groups, wherein each psychopharmacological agent is given to one group only; establishing a cerebral blood flow (CBF) perfusion baseline, blood oxygenation baseline or their combination for all groups, using scanning with arterial spin labeling (ASL) perfusion magnetic resonance imaging (MRI) or absolute T2 mapping MRI; inducing the psychiatric condition, or stress in all groups while individuals in the groups are undergoing scanning with arterial spin labeling (ASL) perfusion magnetic resonance imaging (MRI) or absolute T2 mapping MRI; capturing changes in the cerebral blood flow (CBF), blood oxygenation or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern or a combination thereof between the individuals in each group with a reference database, wherein the reference database is taken from healthy individual or pool of individuals under similar stress-inducing conditions, and wherein cerebral blood flow perfusion pattern, blood oxygenation pattern or a combination thereof, taken of the group which most resemble the cerebral blood flow perfusion pattern, blood oxygeantion pattern or their combination, of the reference database, is the optimal anxiolytic drug for the targeted psychiatric condition.

### BRIEF DESCRIPTION OF THE DRAWINGS

FIG. 1 shows stress-eliciting paradigm.

FIG. 2 shows the average subjective ratings of stress and anxiety, heart rate, and salivary-cortisol level during the time course of the stress experiment. Time 0 indicates the start of MRI experiments. The yellow columns represent the perfusion fMRI scans (each 8 min) and the dark green column represents the anatomical scan. Behavioral ratings and salivary-cortisol samples were taken between scans, whereas heart rate was continuously recorded every 2 min. Note that the peak in salivary-cortisol level lags behind other measures. The error bars indicate standard error.

FIG. 3 shows three-dimensional rendering of the regression-analysis results, which use the CBF change during stress tasks (high-stress_low-stress task) (A) or the CBF change at baseline (baseline 2_baseline 1) (B) as the dependent variable and the change in perceived stress from the low- to high-stress task as the predictor. Also shown are scatterplots of changes in CBF during stress tasks (C) and at baseline (D) as a function of changes in perceived stress between the two stress tasks. Each data point represents one subject. Mean CBF values are drawn from the ROI defined by the activation cluster. Brain regions showing significant association with perceived stress include: right prefrontal cortex (RPFC), left insula/Putamen (LIn/Pu), right insula/putamen (RIn/Pu), anterior cingulate cortex (ACC).

FIG. 4 shows three-dimensional rendering of the regression-analysis results, which use the CBF change at baseline (baseline 2−baseline 1) as the dependent variable and the AUC measures of salivary-cortisol level (A) or the change in heart rate from the low- to high-stress task (B) as the predictor. Also shown are scatterplots of mean baseline CBF changes as a function of cortisol (C) and heart rate (D) in activation clusters. Brain regions showing significant association with cortisol or heart rate include: right prefrontal cortex (RPFC), right obitofrontal cortex (ROrFC); precuneus (preCun); left angular gyrus (LAG); right angular gyrus (RAG); right frontal cortex (RFC); right inferior temporal cortex (RIT).

FIG. 5 shows three-dimensional rendering of the regression-analysis results, which use the CBF change during stress tasks (high-stress_low-stress task) (A) or the CBF change at baseline (baseline 2_baseline 1) (B) as the dependent variable and the change in perceived anxiety from the low- to high-stress task as the predictor. Anxiety related brain regions include: Left insula/putamen/amygdala (LIn/Pu/Am); right putamen/amygdala/hippocampus (RPu/Am/Hi); right superior temporal cortex (RST), anterior cingulate cortex (ACC).

FIG. 6 shows the mean subjective stress rating, heart rate, and salivary-cortisol level during the time course of the control experiment. None of these measurements shows significant variation across the MR scans based on repeated-measures ANOVA.

FIG. 7 shows axial and sagittal sections of the regression-analysis results, which use the cerebral blood flow (CBF) change during tasks (high-stress task−low-stress task) as the dependent variable and the area under the curve (AUC) measures of salivary cortisol level as the predictor. Anteromedial prefrontal cortex (AMPF)

FIG. 8 shows axial sections of the regression-analysis results, showing consistent right prefrontal cortex (RPFC) activation when CBF within the left homologous region of interest (ROI) was included as a covariate in a general linear model (GLM) (Upper) or when left hemispheric CBF was subtracted from the right hemisphere and used as the dependent variable in the GLM (Lower). Each column represents one type of analysis, with corresponding captions on the bottom.

FIG. 9 shows axial sections of the regression-analysis results, showing the difference in brain-activation patterns associated with perceived stress (Upper) and anxiety (Lower). When perceived anxiety is included as a covariate in the regression model, RPFC activation is still significantly correlated with perceived stress (Right), suggesting that lasting effects of stress cannot be attributed to anxiety. In contrast, when perceived stress is included as a covariate in the regression model, left insula/putamen/amygdala (LIn/Pu/Am) and right superior temporal cortex (RST) activations are still significantly correlated with perceived anxiety (Right), suggesting that CBF changes in these brain regions are specifically associated with anxiety.

FIG. 10 shows Axial and coronal sections of the results from within-subject comparison of CBF between the low- and high-stress tasks. Orange and blue indicate activation and deactivation during serial subtraction relative to the counting-backward condition. Abbreviations: Right insula/putamen (RIn/Pu); anterior cingulate cortex/medial prefrontal cortex (ACC/MPF); precuneus/inferior parietal cortex (preCun/IPC; left inferior temporal cortex (LIT); orbitofrontal cortex (OrF; left prefrontal cortex (LPFC); right angular gyrus (RAG); bilateral deactivation clusters covering pre- and postcentral gyri, superior and middle temporal cortex, and insula.

FIG. 11 shows Axial and coronal sections of the results from within-subject comparison of CBF between the two baseline conditions. Abbreviations: Thalamus (Th); left prefrontal cortex (LPFC); posterior cingulate cortex (PCC); left inferior temporal cortex (LIT); left superior temporal cortex (LST).

FIG. 12 Average subjective ratings of stress, anxiety, heart rate and salivary cortisol level during the time course of the stress experiment in the male and female group. All the behavioral and physiological measures are significantly increased after the high stress task. The error bars indicate standard error.

FIG. 13. Axial sections of regression analysis results performed in the male and female group respectively, showing consistent RPFC activation in all the three analyses performed in males. These analyses use the CBF change during stress tasks (high stress−low stress task) (A) and the CBF change at baseline (baseline 2−1) (B) as the dependent variable, and the change in perceived stress from the low to high stress task as the independent variable. Additional analyses use the CBF change at baseline (C) as the dependent variable, and AUC measures of salivary cortisol as the independent variable. Scatter plots of corresponding CBF changes in RPFC ROI (indicated by white circles) as a function of perceived stress or AUC measures of salivary cortisol are displayed.

FIG. 14. Axial sections of regression analysis results performed in the male and female group respectively, showing consistent deactivation of left orbitofrontal/inferior frontal cortex (LOrF/IFC) in all the three analyses performed in males. The analyses are the same as shown in FIG. 13. Scatter plots of corresponding CBF changes in LOrF/IFC ROI (indicated by white circles) as a function of perceived stress or AUC measures of salivary cortisol are displayed.

FIG. 15. Axial sections of regression analysis results performed in the male and female group respectively, showing limbic and cingulate activation only in females. The analyses are the same as shown in FIG. 13. Scatter plots of corresponding CBF changes in ventral striatum and dorsal ACC (dACC) ROIs (indicated by white circles) as a function of perceived stress or AUC measures of salivary cortisol are displayed.

FIG. 16. Three-dimensional rendering of the results comparing the mean acute (High−Low stress task) and lasting (Baseline 2−1) CBF responses between the male and female groups. The greater right-sided male activation during tasks (A) and greater left-sided female activation at baseline (B) are shown. Also shown are diamond plot of changes in RPFC CBF from the low to high stress task (C) (93.8% separation), and scatter plot of the support-vector-machine (SVM) scores for classification of female and male stress responses based on CBF changes in 4 ROIs of RPFC, LOrF, dACC and LIn (D) (100% separation).

### DETAILED DESCRIPTION OF THE INVENTION

This invention relates in one embodiment to the use of a quantitative functional MRI (fMRI)—arterial spin-labeling perfusion MRI or absolute T2 mapping MRI in the non-invasive neuroimaging of a subject's brain in response to stress-inducing psychological stimuli.

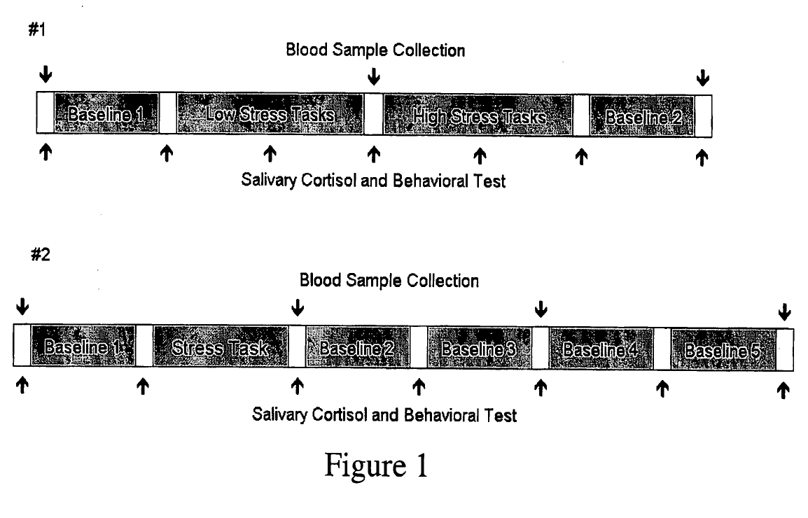

In one embodiment, a particular fMRI technology, termed arterial spin labeling (ASL) perfusion MRI, is used to measure dynamic variations in cerebral blood flow during an experimental stress paradigm (see FIG. 1). In one embodiment, the term MRI refers to magnetic resonance imaging. Magnetic resonance imaging refers in one embodiment to a technique for magnetically exciting nuclear spins of a subject placed in a static magnetic field by applying a radio-frequency signal with the Larmor frequency, and obtaining images using FID (free-induction decay) signals or echo signals induced with the excitation. One category of the magnetic resonance imaging is ASL (Arterial Spin Labeling) imaging. This imaging provides perfusion (tissue blood) images in which blood vessels and microcirculation of a subject are reflected, without injecting contrast medium into the subject, i.e., non-invasively. In one embodiment, the ASL method used in the methods described herein, includes a “continuous ASL (CASL) technique” or a “pulsed ASL (PASL) technique” or a “pseudo-continuous ASL technique”. CASL technique refers in one embodiment, to a way of applying a largely continuous adiabatic RF wave, while in another embodiment, PASL technique refers to the application of a pulsed adiabatic RF wave that can easily be practiced by a clinical MRI system. In another embodiment, pseudo-continuous ASL technique refers to the application of a train of pulsed RF wave to simulate the effect of CASL.

In one embodiment, a quantitative fMRI technology, termed absolute T2 mapping MRI, is used to measure dynamic variations in magnetic transversal relaxation times (T2 or T2*) during an experimental stress paradigm (see FIG. 1). Transversal relaxation times (T2 or T2*) are directly related to regional magnetic field homogeneity (magnetic susceptability) and provide a quantitative index of the blood oxygen content (blood oxygenation). Measurements of transversal relaxation times require at least two MRI measurements acquired at different echo times, which are modeled by exponential decay of the MRI signal with the time constant of the transversal relaxation time. The term each time refers to the time at which a gradient echo or spin echo is formed in MRI signal.

In one embodiment, the invention provides a method for differentiating a subject's reactivity to psychological stress comprising: establishing a cerebral blood flow (CBF) perfusion or blood oxygenation baseline for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF) or blood oxygenation in brain regions associated with stress responses, wherein the changes are captured during the scanning with arterial spin labeling (ASL) perfusion magnetic resonance imaging (MRI) or absolute T2 mapping MRI; and comparing the captured changes in blood flow or blood oxygenation pattern with changes in blood flow or blood oxygenation pattern in a reference database, wherein the reference database indicates reactivity to psychological stress of a predetermined individual or pool of individuals.

In one embodiment, quantitative functional MRI (fMRI) technique, arterial spin-labeling perfusion MRI is used to elucidate the central circuitry of psychological stress. In one embodiment, cerebral blood flow (CBF) is measured directly by using arterial blood water as an endogenous contrast agent. In one embodiment, perfusion fMRI for use in the methods as described herein, is ideal for imaging a sustained behavioral state, such as stress, with excellent reproducibility over long-term time periods and minimal sensitivity to magnetic-field inhomogeneity effects, that involves the function of deep brain structures. In another embodiment, perfusion fMRI allows ecological paradigms to be used in the MR scanner to induce “natural” stress, due to its reduced scanner noise level or reduced sensitivity to the subject's motion. In one embodiment, absolute T2 mapping MRI for use in the methods as described herein, is ideal for imaging sustained behavioral states with excellent reproducibility over long-term time periods and a high sensitivity to magnetic susceptability effects arising from blood oxygenation changes.

In one embodiment, stress eliciting paradigms are used to induce stress in the methods described herein. As shown in the FIG. 1, two typical paradigms are displayed. Paradigm I includes two baseline conditions, one low stress task such as counting backward in one embodiment, one high stress task such as serial subtraction in another embodiment. Before and after each scan, saliva samples (using a cotton swab placed in the mouth), blood samples and subjective ratings of stress in one embodiment, or anxiety, fatigue, depression or their combination are collected. Throughout the procedure, heart rate is recorded every predetermined period, based on a pulse-oxymetry reading. During the high stress task and in one embodiment, subjects are instructed to perform challenging serial subtraction and respond verbally. In another embodiment, subjects are prompted for faster performance and required to restart the task if an error occurs, providing an element of harassment. In one embodiment, the performance referring to the number of errors and successful subtractions, are recorded. Other embodiments include motivated performance tasks such as multi-tasking is also used to elicit stress using a computerized program. Other embodiments include performance and time pressure, negative psychosocial feedback or their combinations provided to the subjects to induce stress.

In another embodiment, paradigm II is used in the methods described herein to elicit the stress. In one embodiment as shown in FIG. 1, candidates are required to step out the MRI scanner and perform a public speech task, a verbal interactivation task or their combinations between baseline perfusion scans. This design is building on the excellent repeatability of perfusion MRI and absolute T2 mapping MRI even when repositioning of subjects' head is involved. In one embodiment, both public speech and mental arithmetic are proven tasks for inducing psychological stress. Baseline perfusion or T2 mapping scans are performed in one embodiment, to record the lasting effect of stress after the task is completed.

In one embodiment, cerebral blood flow or blood oxygenation changes are extracted to and captured using the methods described herein. In another embodiment, analyses of the perfusion fMRI or T2 map data include head motion correction, co-registration with anatomical MRI, generation of perfusion or T2 maps and normalization to a canonical space. In one embodiment, the main variables measured, are the blood flow or blood oxygenation change from the low to high stress task (paradigm I) and the blood flow or blood oxygenation change pre and post stress tasks (paradigm I and II). The most important brain region of interest is the right prefrontal cortex (RPFC). Other brain regions of interest involve in the stress network include the left prefrontal cortex, left orbitofrontal cortex, anterior cingulate cortex (ACC), insula, puteman, amygdala, striatum, nucleus accumbens (NA) and hippocampus.

In one embodiment, RPFC activation is specifically associated with psychological stress, and this activity persists even beyond the stress-task period. This mapping between behavioral/physiologic state and neuroanatomy is supported by the association of RPFC CBF changes with both subjective and objective measures of stress responses. In another embodiment, difficulty or effort did not contribute to RPFC brain activation. In one embodiment, lasting effects of right prefrontal activation exist during baseline conditions without any induced cognitive task, excluding in another embodiment, potential confounding effects due to cognitive differences between the two stress tasks.

In one embodiment, activation of left insula/putamen (LIn/Pu) region during stress tasks, has been linked with the processing of certain forms of negative affect, especially disgust. In another embodiment, the persistence of the RPFC activation, even after completion of stress tasks, reflects a prolonged state of heightened vigilance and emotional arousal elicited by stressors using the paradigms described herein. In another embodiment, both the ACC, an important region involved in the attentional processing of emotion, and the right insula/putamen regions have sustained activation after stress tasks. In one embodiment, the RPFC activation and, in its unexpected lasting effect, is uniquely associated with psychological stress and is not attributed to emotional responses, including anxiety, frustration or their combination. In another embodiment, the brain regions associated with anxiety such as the insula, or putamen, amygdala, ACC, or their combination in other embodiments, are consistent with existing understanding of emotional networks, supporting the sensitivity and validity of perfusion fMRI according to the methods described herein. In one embodiment, the lasting effect of stress indicates that perfusion fMRI is more suitable approach than the blood-oxygen-level-dependent (BOLD) contrast to study the neural substrates of psychological stress, because subjects could no longer return to a “baseline” state after stress tasks, as assumed in a conventional block design in BOLD fMRI. Therefore, in one embodiment, fMRI as described in the methods of the invention, is used to replace BOLD contrast studies.

In one embodiment, the brain regions used in the methods for differentiating a subject's reactivity to psychological stress; or screening candidates for a high-stress position; or diagnosing a mental disorder associated with a subject's susceptibility to psychological stress; or testing a candidate drug as an anxiolytic drug; or optimizing an anxiolytic drug for a psychiatric condition in other embodiments, which is associated with stress are the right prefrontal cortex (RPFC), left prefrontal cortex, left orbitofrontal cortex, anterior cingulate cortex (ACC), insula, puteman, amygdala, striatum, nucleus accumbens (NA) and hippocampus or a combination thereof.

In one embodiment, the step of inducing stress in the subject, according to the methods for differentiating a subject's reactivity to psychological stress; or screening candidates for a high-stress position; or diagnosing a mental disorder associated with a subject's susceptibility to psychological stress; or testing a candidate drug as an anxiolytic drug; or optimizing an anxiolytic drug for a psychiatric condition in other embodiments, comprises making the subject perform psychomotor vigilance task (PVT); probed recall memory (PRM); visual memory task (VMT); synthetic workload task (SYNW); meter reading task (MRT); logical reasoning task (LRT); Haylings sentence completion (HSC), mirror tracing, Stroop tasks, challenging arithmetical tasks, public speaking, interviews or verbal interaction, challenging or unsolvable anagram, solving puzzles, imagining or recalling dysphoric or stressful experiences, watching disturbing or fearful video or pictures, listening to depressing or noisy audio, or a combination thereof.

The term psychomotor vigilance task (PVT), refers in one embodiment to psychomotor vigilance task (PVT), which requires subjects to sustain attention and respond to a randomly appearing light on a computer screen by pressing a button. PVT performance lapses refer to the times when a subject failed to respond to the task in a timely manner (i.e., <500 msec.); lapses are recorded in one embodiment, each minute throughout the test and then totaled for the duration of the test. In one embodiment, Stroop tasks demonstrate interference in the reaction time of a task, often when a word such as blue, green, red, etc. is printed in a color differing from the color expressed by the word's semantic meaning. In another embodiment, verbal interaction tasks involve participants to verbally interact with an experimenter, or confederate, or another participant in other embodiments.

Probed recall memory (PRM), refers in another embodiment, to the memorisation of a number of pairs of unrelated words in a short time period and subsequent recall of word pairing a predetermined time later. Visual memory task (VMT), requires in one embodiment the subjects to remember and replicate positions of a continually increasing sequence of flashing blocks. In another embodiment, synthetic workload task (SYNW), refers to a multicognitive task comprising four tasks completed simultaneously on a split screen, including probed memory, visual monitoring and simple auditory reaction time. Meter reading task (MRT) refers to a numerical memory and recall task; Working memory task (WMT) requires the subjects to determine whether the target stimulus is the same or different from a previously displayed cue stimulus; Logical reasoning task (LRT) involves attention resources, decision-making and response selection; Haylings sentence completion task (HSC) involves subjects completing a sentence with a single word that is either congruous or incongruous with the overall meaning of the sentence. In one embodiment, the term “Anagram” refers to rearranging the letters of a word or phrase to produce other words, using all the original letters exactly once.

In another embodiment, public speaking tasks involve participants prepare and deliver a speech on an assigned topic; Interviews require participants to discuss a personal topic such as a negative life experience or an aspect of their personality; Marital conflict interactions, in which couples discuss a problem in their relationship. Emotion induction procedures include in one embodiment the presentation of emotion-eliciting material designed to automatically elicit a negative affective state (e.g., film), as well as free or guided mental generation of emotional states, in which participants recall a situation in which they felt a specific affective state, acted out an emotional scenario, or experienced the mood described by a series of statements. In noise exposure tasks, participants experience either intermittent or continuous loud noise.

In one embodiment, time pressure, performance monitoring, negative psychosocial feedback or their combinations are provided to the subjects to induce robust stress responses. In another embodiment, the tasks described above are modified to have reduced work load and difficulty as the control condition or low stress tasks. In another embodiment, no time or performance pressure is involved during the control condition or low stress tasks.

In one embodiment, the according to the methods for differentiating a subject's reactivity to psychological stress; or screening candidates for a high-stress position; or diagnosing a mental disorder associated with a subject's susceptibility to psychological stress; or testing a candidate drug as an anxiolytic drug; or optimizing an anxiolytic drug for a psychiatric condition in other embodiments, further comprise the steps of collecting additional data between the steps of establishing a baseline and the step of inducing stress; between the step of inducing stress and the step of imaging cerebral blood flow (CBF) or T2 map changes; and after the step of imaging cerebral blood flow (CBF) or T2 map changes.

In one embodiment, the joint correlations of baseline CBF changes in the RPFC with perceived stress, cortisol, and heart rate indicate that sustained regional brain activation after stressors according to the methods described herein, are a characteristic feature of stress. The time scale of the acute stress response, including its lasting effect, is an important issue in the neurobiology of stress. After a moderately acute stressor, in one embodiment, it take minutes for heart rate and 1-2 h for cortisol to return to the baseline, although behavioral ratings may recover faster. In another embodiment, a mild-to-moderate stressor, causes elevation in peripheral cortisol that peakes at about 10 min after the high-stress task. Given the temporal coincidence of RPFC CBF increase and stress-hormone elevation, in another embodiment, cortisol might be a mediator of the lasting effect of central stress response.

Deception has major legal, political and business implications. Thus, there is a strong general interest in repeatable and quantitative methods for determining with a high degree of certainty when one is actively involved in deception. In one embodiment, deception of another individual is the intentional negation of subjective truth, suggesting that in another embodiment alteration of truthful response is a prerequisite of intentional deception. In another embodiment, the term “deception” or “intentional deception,” refers to an act intended to create in the mind of the individual being deceived, a perception of reality which is different from the individual causing the deception, and in yet another embodiment, different from objective reality.

In one embodiment, conscious deception is associated with observable stress responses such as galvanic skin response, heartbeat rate, and blood pressure, as well as alternations of neural activity in brain regions associated with stress responses. In one embodiment, regional brain activity in the deceiving individual, as elicited by that individual's inhibition, alteration of augmentation of the truth response, is captured using the individual's changes in the cerebral blood flow (CBF), blood oxygenation, or their combination in brain regions associated with stress, such as the RPFC, cingulate cortex, insula and amygdala in certain embodiments. In another embodiment, the above method relying on detecting changes in neural activity in stress related brain regions is combined with brain imaging methods that rely on detecting alternations in neural activity in brain regions associated with cognitive control, inhibition and alternation of truth, to improve the accuracy for determining when one is actively involved in deception.

In one embodiment, a reduction in CBF in ventrolateral left prefrontal cortex and left orbitofrontal cortex, occurs simultaneously with activation of the RPFC in subjects experiencing stress, within-subject comparison of CBF between the high- and low-stress conditions. These latter areas, in conjunction with ventral striatum, subserve in another embodiment, the positive-emotion network and reward system that mediates approach-related, appetitive goals. The changes indicate in one embodiment, an inhibition of brain regions supporting appetitive and hedonic goals during psychological stress. In one embodiment, the stress-related brain regions used for capturing data according to the methods described herein are also associated with negative emotions, including right insula and putamen, during the high-stress relative to the low-stress task. The observed activation in the dorsomedial prefrontal cortex/ACC and precuneus/parietal cortex reflects in one embodiment mental arithmetical performance or assessment of the mental state during the serial-subtraction task, whereas the CBF reduction in pre- and post central gyri and temporal cortex reflect in another embodiment, a more frequent verbal movement and greater auditory stimulation during counting backward versus serial subtraction.

In one embodiment, the additional data collected in conjunction with the CBF or blood oxygenation changes in stress-related brain regions, used in are saliva samples, blood samples, heart rate, blood pressure, skin conductance and subjective ratings of the methods for differentiating a subject's reactivity to psychological stress; or screening candidates for a high-stress position; or diagnosing a mental disorder associated with a subject's susceptibility to psychological stress; or testing a candidate drug as an anxiolytic drug; or optimizing an anxiolytic drug for a psychiatric condition in other embodiments, is stress, or anxiety, fatigue, depression or a combination thereof in other embodiments.

In one embodiment, regional brain activity associated with both behavioral and physiological stress responses is captured by using perfusion fMRI. The localization of brain regions related to emotion, vigilance, and goal-directed behavior within the RPFC indicates that this region serves a central role in coordinating a range of biological and behavioral responses to stress.

In one embodiment, the reference database used in the methods for differentiating a subject's reactivity to psychological stress; or screening candidates for a high-stress position; or diagnosing a mental disorder associated with a subject's susceptibility to psychological stress; or testing a candidate drug as an anxiolytic drug; or optimizing an anxiolytic drug for a psychiatric condition in other embodiments, comprises the captured image of CBF or blood oxygenation changes taken from the proper brain regions associated with stress of a predetermined subject or pool of subjects. In another embodiment, those brain regions are the brain regions described herein.

In one embodiment, the predetermined subject or pool of subjects is selected from top executives, elite athletes, performers, astronauts, air traffic controllers, combat soldiers, political leaders, or a combination thereof. A reference database of brain activation to stress is built in one embodiment, based on a large pool of normal subjects and elites. Classification of the database into categories of high and low stress responders is realized by regression of brain activation with existing standards for assessing subjects' stress responses. These include heart rate, blood pressure, cortisol and other psychological testing results. This is accomplished in one embodiment, with uni-variable linear regression (general linear model). In another embodiment, brain activation templates are developed using automatic multivariable clustering/regression, or neural network classification. These methods simultaneously take into account cerebral blood flow or blood oxygenation changes in multiple brain regions. In one embodiment, candidates are differentiated by comparison of cerebral flow or blood oxygenation change or both in given brain regions (e.g. RPFC) with those of the templates developed, by calculating the minimal distance between the sample and templates. In another embodiment, multi-variable classification or fuzzy logic are also used to identify candidates that mirror stress reaction of elites; or in another embodiment, of paranoid schizophrenics, or drug addicts, depressives, phobics, subjects afflicted with obesity, hypertension, diabetes, obsessive compulsive disorder, post-traumatic stress syndrome, or a combination thereof in other embodiment. In one embodiment, the methods described herein are used to differentiate candidates with brain activation to stress matching those of the subject or pool of subjects selected; or with brain activation to stress dissimilar to those of the subject or pool of subjects in another embodiment.

Gender difference in stress response has been characterized by “fight-or-flight” in men and “tend-and-befriend” in women. Men are generally more vulnerable to the adverse health effects of stress, including hypertension, aggressive behavior, or abuse of alcohol or drugs. Women, on the other hand, have a twice high rate of depression and anxiety disorders compared to men. According to this aspect of the invention, and in one embodiment, the methods of the invention are used for differentiating a subject's reactivity to psychological stress; or screening candidates for a high-stress position; or diagnosing a mental disorder associated with a subject's susceptibility to psychological stress; or testing a candidate drug as an anxiolytic drug; or optimizing an anxiolytic drug for a psychiatric condition in other embodiments, are age and gender specific.

In one embodiment, men have greater acute stress response manifested as RPFC activation, whereas the lingering stress effect is stronger in women particularly in emotion related brain regions (ACC). In one embodiment, regression analyses of CBF data with subjective ratings of stress and salivary cortisol changes consistently show RPFC activation and left orbitofrontal/inferior frontal cortex (LOrF/IFC) suppression in male subjects. In another embodiment, regression analyses of CBF data with subjective ratings of stress and salivary cortisol changes consistently show limbic activation including ACC, insula and putamen in female subjects. In one embodiment, linear classification method using support-vector-machine (SVM) is able to differentiate male and female stress responses with an accuracy of 94%, based on single region of the RPFC in one embodiment. In another embodiment, support-vector-machine (SVM) achieves a perfect separation (100%) of male and female brain activation to stress based on 4 brain regions of: RPFC, left insula, left orbitofrontal cortex and ACC.

In another embodiment, the methods described hereinabove are used in the methods described herein. In one embodiment, the invention provides a method of screening candidates for a high-stress position comprising the steps of: establishing a cerebral blood flow (CBF) perfusion or blood oxygenation baseline for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF) or blood oxygenation or bith in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern with changes in blood flow or blood oxygenation pattern in a reference database, wherein the reference database indicates reactivity to psychological stress of a predetermined individual or pool of individuals proven as appropriate for the high-stress position sought to be screened for.

In another embodiment, the inducing stress according to the methods of screening candidates for a high-stress position comprises inducing stress typical of the position for which the screening is sought. In one embodiment, the subject is an astronaut and following the eliciting of motion sickness, the subject is asked to identify which hand of a mannequin is holding a certain symbol. The mannequin may be upside down, sideways, or backwards so candidates have to adjust their minds accordingly. In one embodiment, the predetermined subject or pool of subjects is a top executive, or an elite athlete, a performer, an astronaut, an air traffic controller, a combat soldier or pilot, a political leader, or a combination thereof in other embodiments.

In another embodiment, the subject or pool of subject used for any embodiment of the methods described herein, are paranoid schizophrenics, drug addicts, depressives, phobics, subjects afflicted with obesity, hypertension, diabetes, obsessive compulsive disorder, autism, panic attacks, post-traumatic stress syndrome, or a combination thereof and the like.

In one embodiment, the invention provides a method of diagnosing a mental disorder (referring in another embodiment, to enduringly deviating patterns of perceiving, relating to, and thinking about the environment and oneself that are exhibited in a wide range of social and personal contexts), associated with a subject's susceptibility to psychological stress comprising the steps of establishing a cerebral blood flow (CBF) perfusion or blood oxygenation baseline for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF) or blood oxygenation in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern with changes in blood flow or blood oxygenation pattern or both flow and oxygenation patterns in another embodiment, in a reference database, wherein the reference database indicates reactivity to psychological stress of a predetermined individual or pool of individuals correctly diagnosed with said mental disorder sought to be diagnosed.

In one embodiment, the mental disorder being diagnosed is post-traumatic stress disorder (PTSD), obsessive-compulsive disorder (OCD), and social anxiety disorder (social phobia) (SAD), and the like. In one embodiment, Post-Traumatic stress disorder patients exhibit disruptions in the neurological pathways associated with fear, as expressed in the ascending serotonin pathway, originating in the dorsal raphe nucleus and innervating the amygdala and frontal cortex, thereby facilitating conditioned fear. In another embodiment, the dorsal raphe nucleus-periventricular pathway inhibits inborn fight-or-flight reactions to impending danger; and in yet another embodiment, the pathway connecting the median raphe nucleus to the dorsal hippocampus promotes resistance to chronic, unavoidable stress. In one embodiment, serotonin terminals which are interrupted in PTSD patients, from the dorsal raphe and norepinephrine terminals from the locus ceruleus, converge on the amygdala to mediate fear responses. In one embodiment, PTSD patients will exhibit changes in CBF that are typical among PTSD patients, and that are different than normal subjects under similar stressful circumstances. In another embodiment, these changes in CBF are expressed in the amygdale as described herein, and are capable of being diagnosed according to the methods of the invention.

In one embodiment, the anterior cingulate cortex (ACC) have been implicated in a number of psychiatric disorders, such as schizophrenia in one embodiment, or obsessive-compulsive disorder, depression, post-traumatic stress disorder, or autism in other embodiments. In another embodiment, schizophrenia, or obsessive-compulsive disorder, depression, post-traumatic stress disorder, or autism patients in other embodiments will exhibit changes in CBF that are typical among schizophrenia OCD, depression, PTSD, or autism in other embodiments, and that are different than normal subjects under similar is induced stress. In another embodiment, these changes in CBF or blood oxygenation or both, are expressed in the ACC as described herein, and are capable of being diagnosed according to the methods of the invention. In another embodiment, patients with panic disorder show significant CBF increases bitemporally and CBF increases in the anterior cingulate gyrus, the claustrum-insular-amygdala region and in the cerebellar vermis, when compared with non-panickers, making the methods of the invention described herein, uniquely capable of diagnosing in one embodiment, or evaluating medication and it's efficacy in other embodiment, the mental illnesses exhibiting differentiated CBF from normal subjects.

In one embodiment, the methods described in any embodiment hereinabove, are used to obtain the images captured and used to generate the library of images described herein. In another embodiment, the invention provides a library of images of cerebral blood flow changes or blood oxygenation changes, or in another embodiment both flow and oxygenation changes in brain regions associated with stress response, wherein the images are captured in response to psychological stress, using scanning with arterial spin labeling (ASL) perfusion magnetic resonance imaging (MRI), or absolute T2 mapping MRI taken from a predetermined subject or pool of subjects.

In one embodiment, the invention provides a machine readable media comprising a library of images of cerebral blood flow or blood oxygenation changes or both, in brain regions associated with stress response, wherein the images are captured in response to psychological stress, using scanning with arterial spin labeling (ASL) perfusion magnetic resonance imaging (MRI) or absolute T2 mapping MRI, taken from a predetermined subject or pool of subjects, which are captured in another embodiment by any embodiment described herein or its equivalent. In one embodiment “machine readable media”

In one embodiment, the invention provides a method of testing a candidate drug as an anxiolytic drug, comprising the step of: deviding a cohort of healthy subjects into two groups, administering to one group a placebo and to the other group the candidate drug; establishing a cerebral blood flow (CBF) perfusion baseline or blood oxygenation baseline for both groups, or both perfusion and oxigenation, using scanning with arterial spin labeling (ASL) perfusion magnetic resonance imaging (MRI) or absolute T2 mapping MRI; inducing stress in both groups, while individuals in the groups are undergoing scanning with arterial spin labeling (ASL) perfusion magnetic resonance imaging (MRI) or absolute T2 mapping us MRI; capturing changes in the cerebral blood flow (CBF) or blood oxygenation in brain regions associated with stress responses, wherein the changes are captured during the scanning with arterial spin labeling (ASL) perfusion magnetic resonance imaging (MRI) or absolute T2 mapping MRI; and comparing the captured changes in blood flow or blood oxygenation pattern between the individuals in the group that received placebo, with the individuals in the group that received the candidate drug, wherein blood flow or blood oxygenation pattern in the group which received the candidate drug, which yields cerebral blood flow or blood oxygenation pattern which resembles the baseline cerebral blood flow perfusion or blood oxygenation closer than that of the cerebral blood flow perfusion or blood oxygenation of the group that received placebo, indicate the candidate drug is an anxiolytic drug. In one embodiment, a combination of both blood flow and oxygenation levels are used as the biomarkers scanned using the MRI techniques described in the methods provided herein and in the libraries described herein.

In one embodiment, the step of inducing stress in the methods of screening psychopharmacological agents, or their optimization in other embodiments, comprises inducing stress typical of the stress triggering the psychiatric condition sought to be targeted. In one embodiment, the anxiolytics or psychopharmacological agents sought to be screened or optimized according to the methods described herein are for conditions such as depression, or dementia, night terrors, obsessive-compulsive disorder, panic attacks, or anxiety in other embodiments.

In one embodiment, anxiety, refers to excessive or inappropriate arousal characterized by feelings of apprehension, uncertainty, and fear. In other embodiments, there is no real or appropriate threat to which the anxiety can be attributed. In one embodiment, anxiety can paralyze an individual into inaction or withdrawal. In another embodiment, anxiety is a symptom of other psychologic or medical problems, such as depression, substance abuse, or thyroid disease. In one embodiment, two primary anxiety types are classified. Generalized anxiety disorder (GAD), referring to long-lasting and low-grade, and panic disorder, which has more dramatic symptoms. In another embodiment anxiety disorders refer to phobias, performance anxiety, obsessive-compulsive disorder (OCD), and post-traumatic stress disorder (PTSD).

In one embodiment, the term “anxiolytic” refers to any agent capable of reducing tension, anxiety or agitation in a subject. In one embodiment, the candidate agents or drugs identified by the methods described hereinabove, are used in the methods of evaluating efficiency of such anxiolytics in the methods described herein. In one embodiment, the invention provides a method of optimizing an anxiolytic drug for a psychiatric condition, comprising the steps of: dividing a cohort of subject exhibiting the psychiatric condition for which the psychopharmacological agents are sought to be optimized, to a number of groups equal to the number of psychopharmacological agents sought to be optimized; administering the psychopharmacological agents to the groups, wherein each anxiolytic drug is given to one group only; establishing a cerebral blood flow (CBF) perfusion or blood oxygenation baseline or both flow and oxigenation levels for all groups, using scanning with arterial spin labeling (ASL) perfusion magnetic resonance imaging (MRI) or absolute T2 mapping MRI; inducing the psychiatric condition, or stress in all groups while individuals in the groups are undergoing scanning with arterial spin labeling (ASL) perfusion magnetic resonance imaging (MRI) or absolute T2 mapping MRI; capturing changes in the cerebral blood flow (CBF) or blood oxygenation in brain regions associated with stress responses, wherein the changes are captured during the scanning with arterial spin labeling (ASL) perfusion magnetic resonance imaging (MRI) or absolute T2 mapping MRI; and comparing the captured changes in blood flow or blood oxygenation pattern between the individuals in each group with a reference database, wherein the reference database is taken from healthy individual or pool of individuals under similar stress-inducing conditions, and wherein cerebral blood flow perfusion or blood oxygenation pattern taken of the group which most resemble the cerebral blood flow perfusion or oxygenation of the reference database, is the optimal anxiolytic drug for the targeted psychiatric condition.

In another embodiment, the psychopharmacological agents that are being optimized according to the methods described herein are benzodiazepines, such as alprazolam, or chlordiazepoxide, clonazepam, clorazepate, diazepam, halazepam, lorazepam, oxazepam, and prazepam; non-benzodiazepine agents, such as buspirone; and tranquilizers, such as barbituates, and antidepressant, such as monoamine oxidase inhibitors, tricyclic antidepressants, selective serotonin reuptake inhibitors, and the like in other embodiments. In one embodiment, the term “psychiatric condition” refers to stress-related pathological condition, such as depression in one embodiment, or dementia, sleep disorder, obsessive-compulsive disorder, panic attacks, social phobia, post-traumatic stress disorder (PTSD), or anxiety disorder and their combination in other embodiments.

The following examples are presented in order to more fully illustrate the preferred embodiments of the invention. They should in no way be construed, however, as limiting the broad scope of the invention.

### EXAMPLES

### Materials and Methods

### Subjects

Thirty-two subjects participated in this study. Twenty five subjects (age 24.1±2.8 yrs, 12 female) participated in the stress experiment and 7 subjects (age 23.4±1.3 yrs, 4 female) participated in the control experiment. Two of the 25 subjects participating in the stress experiment were excluded because of incomplete behavioral data and abnormally high baseline salivary cortisol level (>3 SD), resulting in 23 complete data sets (11 female) for stress tasks. All of the subjects were native English speakers and screened for history of neurologic and psychiatric disease. Written informed consent was obtained before all human studies, in accord with an Institutional Review Board approval from the University of Pennsylvania.

### Experimental Procedures:

The mental arithmetic task was adapted as the psychological stressor during perfusion fMRI scans. Subjects were instructed to perform serial subtraction of 13 from a four-digit number and respond verbally. During the task, the subjects were prompted for faster performance and were required to restart the task if an error occurred. This high-stress condition was preceded by a low-stress condition, during which subjects counted aloud backward from 1,000 (to control for activation of verbal and auditory centers). Subjects were given a 15-min resting period after they arrived at the MR facility. The scanning protocol consisted of four perfusion fMRI scans (8 min each) and an anatomical scan (6 min) at the end. During the second and third perfusion fMRI scans, subjects were instructed (at the beginning of each session) to perform the counting-backward (low-stress) and serial-subtraction (high-stress) task. The low- and high-stress scans were conducted in a fixed order to eliminate contamination of the control condition by increased emotional reactivity elicited by the high-stress task. The first and last perfusion fMRI scans were baseline conditions without task.

Self-report of stress and anxiety level (on a scale of 1 to 9) and saliva samples (using a cotton swab placed in the mouth for 2 min) were collected right after the subjects entered the MR scanner and after each MR scan. Subjects were also required to report the level (on a scale of 1 to 9) of effort, frustration, and task difficulty after the low- and high-stress tasks. Throughout the experiment, heart rate was recorded every 2 min, based on a pulse-oxymetry reading. Saliva samples were stored at −80° C. until assayed. To measure stress caused by undergoing MR scanning, a control experiment was conducted using the same scanning protocol, but the subjects were not required to perform any task. Self-report of stress, heart-rate recording, and saliva-sample collection were as described in the stress experiment. All MRI experiments were carried out between 3 p.m. and 5 p.m. to control for diurnal fluctuations in salivarycortisol level.

### Imaging Data Acquisition:

MR scanning was conducted on a 3.0T Trio whole-body scanner (Siemens, Erlangen, Germany), using a standard transmit_receive head coil. A continuous arterial spinlabeling (CASL) technique was used for perfusion fMRI scans. Interleaved images with and without labeling were acquired by using a gradient-echo echo-planar imaging sequence. A delay of 1 sec was inserted between the end of the labeling pulse and image acquisition to reduce transit artifact. Acquisition parameters were field of view (FOV)=22 cm, matrix=64×64, repetition time (TR)=4 sec, echo time (TE)=17 ms, and flip angle=90°. Fourteen slices (6 mm thick with 1.5-mm gap) were acquired from inferior to superior in a sequential order. Each CASL scan with 120 acquisitions took 8 min. A 3D magnetization-prepared rapid gradient echo volumetric scan was used for high resolution T₁-weighted anatomic images: TR=1,620 ms, inversion time (TI)=950 ms, TE=3 ms, flip angle=15°, 160 contiguous slices of 1.0-mm thickness, FOV=192×256 mm2, matrix=192×256, INEX with a total scan time of 6 min.

### Behavioral and Physiological Data Analysis.

The salivary-cortisol level was assayed by using an enzyme immunoassay kit (Salimetrics, State College, Pa.). Behavioral and physiological measurements were analyzed by using repeated-measures ANOVA of the program SPSS 12.0 (SPSS, Chicago) to assess the effect of experimental condition. The differences of the behavioral and physiological measures between the low- and high-stress tasks were entered into a cross-correlation analysis to search for any significant correlation between these measurements across subjects. Because salivary cortisol is a delayed peripheral response, the immediate measurements after stress tasks may not reflect variations in subjects' stress state. Therefore, we measured the area under the curve (AUC) of the salivary cortisol level, calculated as the net area under the stress-response curve (all six samples, see FIG. 1), with reference to the baseline (first sample) by using trapezoidal integration.

### Imaging-Data Analysis

Perfusion fMRI data were analyzed offline by using the program VOXBO (www.voxbo.org) and SPM99 software packages (Wellcome Department of Cognitive Neurology, Institute of Neurology, London). MR image series were first realigned to correct for head movements, coregistered with each subject's anatomical MRI, and smoothed in space with a 3D, 12-mm full-width at half-maximum Gaussian kernel. Perfusion-weighted image series were generated by pairwise subtraction of the label and control images, followed by conversion to absolute CBF image series based on a single compartment continuous arterial spinlabeling perfusion model. Voxel-wise analyses of the CBF data were conducted in each subject by using a general linear model (GLM), including the global time course as a covariate to reduce the effect of spatially coherent noise (first-level analysis). No temporal filtering or smoothing was involved. Two contrasts were defined in the GLM analysis, namely the CBF difference between the two stress tasks (high-stress and low-stress) and the CBF difference between the two baseline conditions (baseline 2−baseline 1).

Individual contrast images (maps for each contrast) were normalized into a canonical space (Montreal Neurological Institute standard brain), and were analyzed by using one-sample t-tests to obtain the activation pattern for the two defined contrasts using a random-effects model that allows population inference (second level analysis). This step provides a within-subject comparison of CBF between corresponding experimental conditions. Furthermore, linear-regression analyses were carried out on these normalized individual maps to obtain the activation pattern correlated with perceived stress and other measurements, by using differences in each of the behavioral and physiological measurements between the high- and low-stress tasks as the independent variable. For salivary cortisol, we used the AUC measurement as the independent variable for regression analyses. Areas of significant activation were identified at the cluster level for the P value <0.005 (uncorrected) and the cluster extent size >94 voxels (2×2×2 mm³), resulting in a cluster-corrected threshold of P<0.05 in SPM⁹⁹. Regions of interest (ROI's) based on activation clusters were generated by using the SPM MARSBAR toolbox. To test the asymmetry of prefrontal activation, the right prefrontal ROI was also flipped in the left-right direction to generate the left homologous ROI. CBF changes of the 23 subjects in these ROI's were extracted and entered into a univariate GLM analysis using the SPSS software to investigate the effect size of each covariate.

### Example 1

### Perfusion Functional MRI Reveals Cerebral Blood Flow Pattern Under Psychological Stress

### Results

### Behavioral and Physiological Data

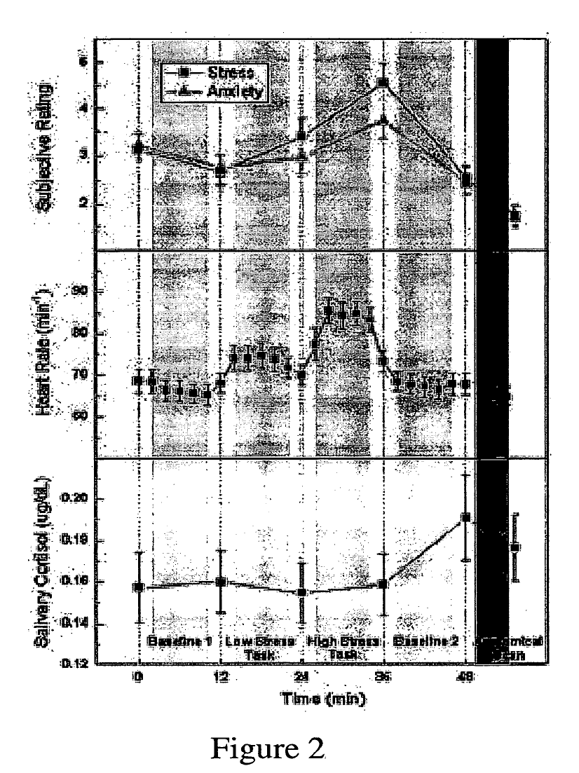

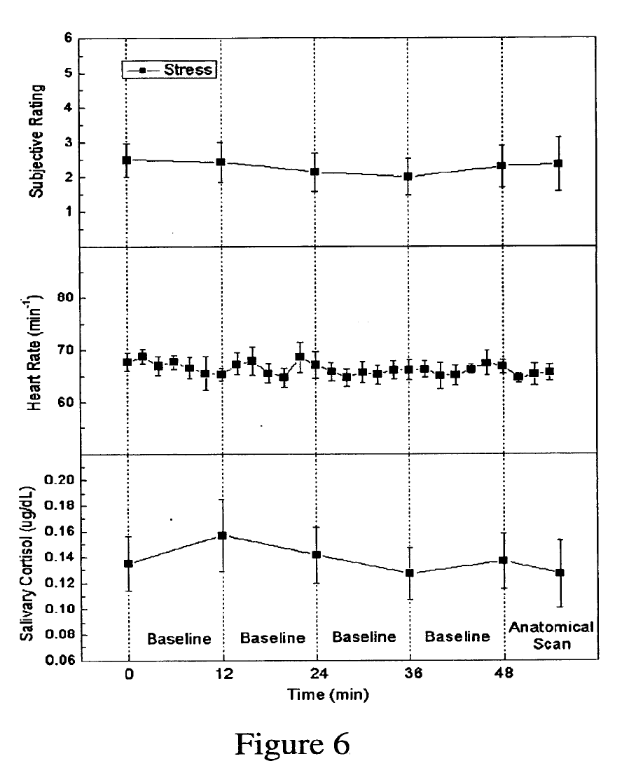

The results of subjects' self-ratings of stress, emotion, and physiological responses suggest that the stress-elicitation paradigm successfully induced a mild-to moderate level of psychological stress. Average self-report of stress (P=0.002) and anxiety (P=0.008) levels and the heart rate (P<0.001) increased from the low-stress task to the high-stress task and decreased during the second baseline period (see FIG. 2). Salivary cortisol, a stress-related hormone, reached its peak 10 min after the end of the high-stress task (P=0.045), consistent with the expected time lag between peripheral cortisol and behavioral measures. Subjects' ratings of task difficulty (P<0.001), effort required (P<0.001), and frustration (P<0.001) were significantly elevated in the high-stress condition relative to the low-stress condition (see Table 1). In addition, we found that perceived stress level was significantly correlated with perceived anxiety level across subjects (r=0.74, P<0.001) and was, to a lesser extent, correlated with perceived frustration (r=0.39, P=0.064). The correlation between self-ratings of task difficulty and effort required also showed a trend toward significance (r=0.40, P=0.057). During the control experiment, none of the behavioral and physiological measures showed significant variation (P=0.12) (see FIG. 6), indicating that undergoing MRI scanning caused little effect on subjects' stress and emotional state.

TABLE 1
Self-report of effort, difficulty, and frustration
during the low- and high-stress tasks (scale 1-9)

| Stress | Effort | Difficulty | Frustration |
|---|---|---|---|
| Low-stress task | 4.4(0.5) | 3.4(0.4) | 3.4(0.4) |
| High-stress task | 7.0(0.3) | 6.6(0.3) | 6.1(0.4) |

Data are presented as mean (standard error)

Imaging Data, Regression Analysis with Perceived Stress.

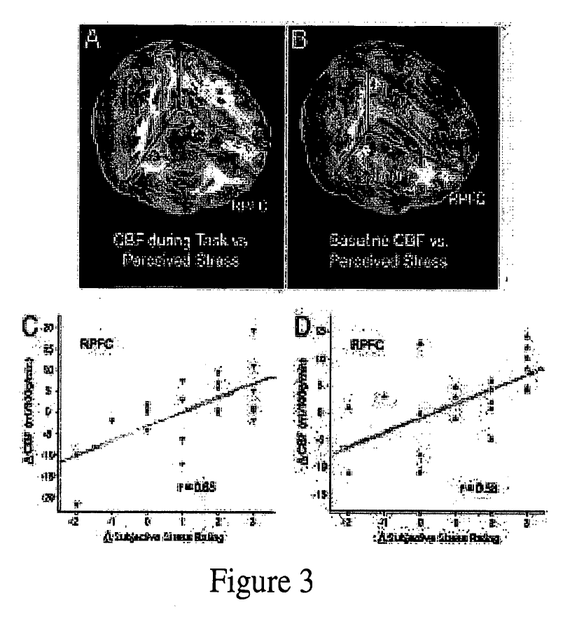

Regression analyses were carried out to search for the specific brain regions associated with individual subject's experience of stress. The hypothesis was that the CBF change induced by the high-stress task compared with the low-stress task should be correlated with the change in perceived stress between these two conditions. A positive correlation was found between the changes in CBF and subjective stress rating in the ventral RPFC (FIG. 3A). A significant correlation was also observed in the left insula/putamen (LIn/Pu) area. The scatterplot of FIG. 3C shows that the serial-subtraction task yielded a greater CBF increment in the ventral RPFC in subjects who reported larger amount of stress elevation. A regression analysis was carried out to determine whether there was any lasting effect of psychological stress on resting state CBF, even after the stressor disappeared. The hypothesis was that the CBF difference between the two baseline conditions (baseline 2_baseline 1) should be correlated with the change in self-report of stress from the low- to high-stress task. Again, a significant correlation was detected between changes in baseline CBF and subjective stress rating during tasks in the ventral RPFC (FIG. 3B). Positive correlations were also observed in the anterior cingulate cortex (ACC) and right insula-putamen. As displayed in the scatterplot of FIG. 3D, greater baseline CBF increment in the ventral RPFC was associated with larger increases in perceived stress during tasks.

Imaging Data, Regression Analysis with Physiological Stress Responses.

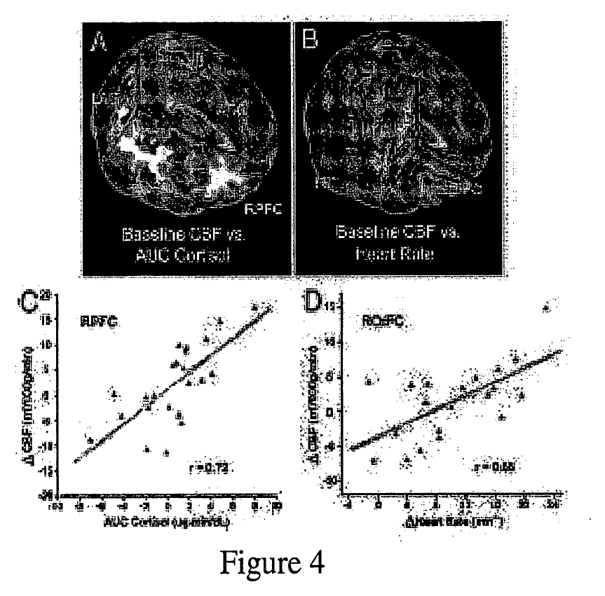

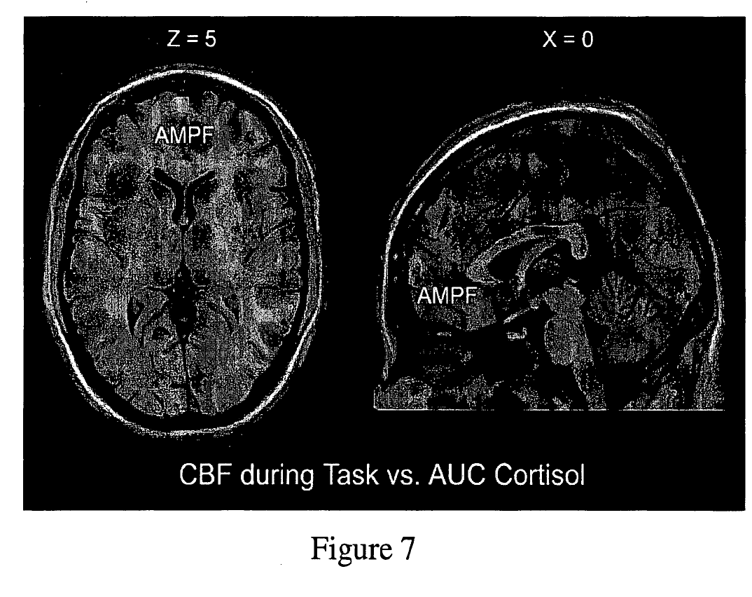

Tests were conducted to estimate whether the observed RPFC activation can be replicated when measures of physiological stress responses were used as the predictor in regression analyses. Changes in baseline CBF pre- and post stress tasks (baseline 2−baseline 1) were found to be significantly correlated with the cumulative salivary cortisol change (AUC measures) in the ventral RPFC (FIG. 4A). FIG. 4A also indicates several other brain regions manifesting significant association between changes in baseline CBF and AUC measures of cortisol, including ACC, and precuneus and left and right angular gyri/inferior parietal cortex. When heart rate was used as the covariate in regression analyses, we found significant associations between variations in baseline CBF (baseline 2−baseline 1) and changes in heart rate from the low- to high-stress task in the right obitofrontal cortex is (ROrFC), dorsolateral right frontal cortex, and right inferior temporal cortex. The scatterplots in FIGS. 4 C and D show that undergoing the two stress tasks yielded a greater increment of baseline CBF in the ventral RPFC and ROrFC in subjects manifesting a larger amount of cumulative salivary-cortisol elevation and greater heart-rate increase from the low- to high-stress task, respectively. However, when regression analyses were performed with CBF differences between the low and high-stress tasks as the dependent variable, we did not observe a significant relationship between RPFC CBF and physiological stress responses. Instead, we found a significant correlation between CBF changes during stress tasks and AUC measures of cortisol in the anteromedial prefrontal cortex (see FIG. 7).

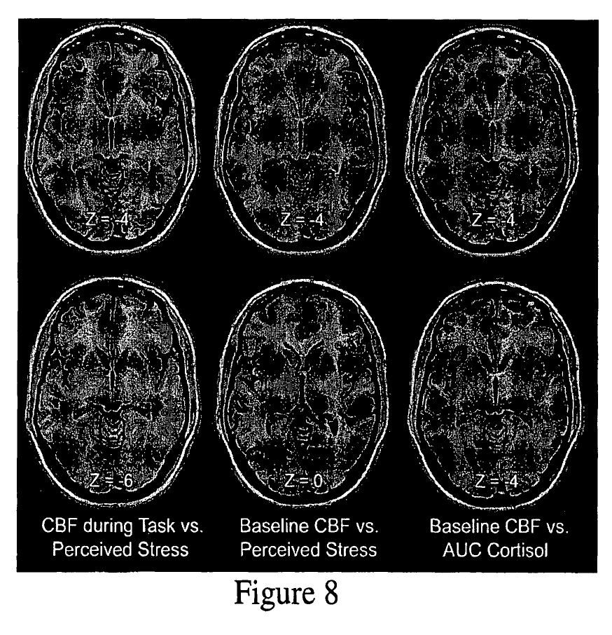

To further test the specificity (asymmetry) of the observed ventral RPFC activation with perceived stress and salivary-cortisol level, regression analyses were repeated by using CBF values derived from a left homologous ROI as a covariate along with subjective stress rating or AUC measures of salivary cortisol. The observed RPFC activation was still significant with left prefrontal CBF included in the regression model. On average, CBF within the left homologous ROI accounted for 17% of the total variance of RPFC CBF, whereas the fractional variance explained by perceived stress and cortisol was 36% and 45%, respectively (P=0.02, Table 2). Furthermore, when the left hemispheric CBF was subtracted from the right hemisphere and used as the dependent variable in the regression analysis, we still observed significant association of ventral RPFC CBF with perceived stress and salivary cortisol (see FIG. 8). These data strongly support the specific association of CBF increase in the ventral RPFC and psychological stress.

TABLE 2
Univariate analysis of variance of baseline CBF changes in ventral RPFC
explained bydifferent covariates and CBF in left homologous ROI

| | | | | | | Heart | | CBF |
|---|---|---|---|---|---|---|---|---|
| Stress | Anxiety | Frustration | Effort | Difficulty | Cortisol | Rate | LPFC | Model |
| 0.363 | 0.028 | 0.011 | 0.058 | 0.073 | 0.453 | 0.032 | 0.171 | 0.753 |
| P = 0.014 | | | | | P = 0.004 | | P = 0.112 | P = 0.003 |

Results are based on values of partial R squared in SPSS, and the model includes all covariates and an intercept. LPFC, left prefrontal cortex

Imaging Data, Regression Analysis with Anxiety and Other Behavioral Measures.

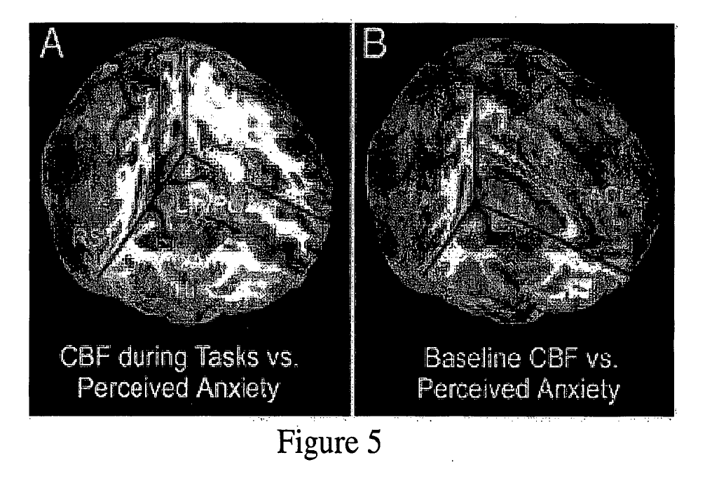

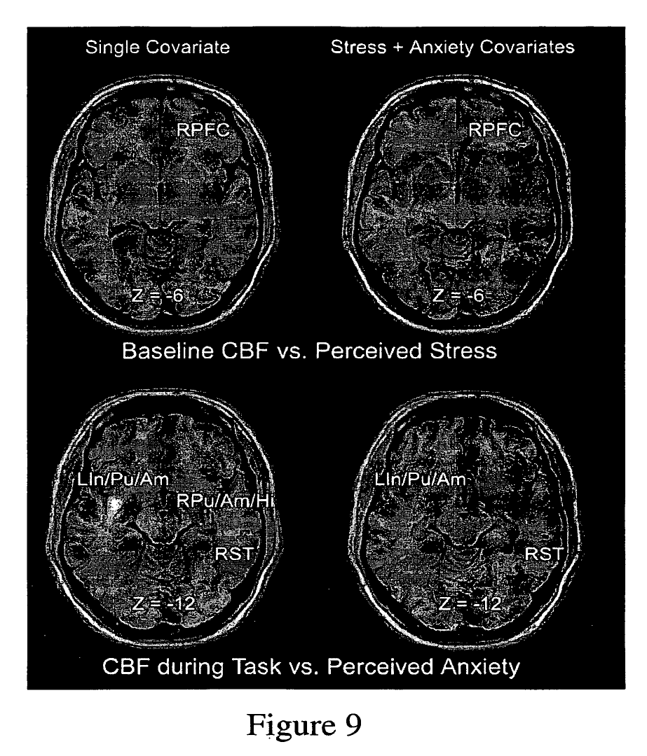

Regression analyses were also repeated with subjective anxiety rating as the independent variable. A strong correlation between the changes in CBF and subjective anxiety rating during stress tasks (high-stress task−low-stress task) was observed in a large activation cluster covering left insula/putamen/amygdala (Lin/Pu/Am) and superior temporal regions. Positive correlations between changes in CBF and perceived anxiety level during stress tasks were also evident in right putamen, amygdale, hippocampus, and right superior temporal regions (FIG. 5A). A positive correlation between changes in baseline CBF (baseline 2−baseline 1) and subjective anxiety rating during stress tasks was observed in ACC (FIG. 5B). The brain activations associated with perceived anxiety partially overlap those related to perceived stress, consistent with our behavioral data showing a correlation between these two variables. However, RPFC CBF, either during stress tasks or at baseline, was not found to vary with changes in subjective anxiety rating. Further regression analyses indicated that baseline CBF change in the ventral RPFC was correlated with perceived stress, even with perceived anxiety included as a covariate in the regression model (see FIG. 9). Table 2 shows that perceived anxiety, when included with perceived stress and other covariates in the GLM, accounted for little variation in ventral RPFC CBF (<3%). In contrast, significant associations between CBF and anxiety ratings during stress tasks could be observed in LIn/Pu/amygdala and right superior temporal regions when perceived stress was included as a covariate in the regression model. Although behaviorally correlated, perceived stress and anxiety seem to be associated with distinguishable brain-activation patterns which overlap in LIn/Pu and ACC.

Imaging Data, within-Subject Comparison of CBF.

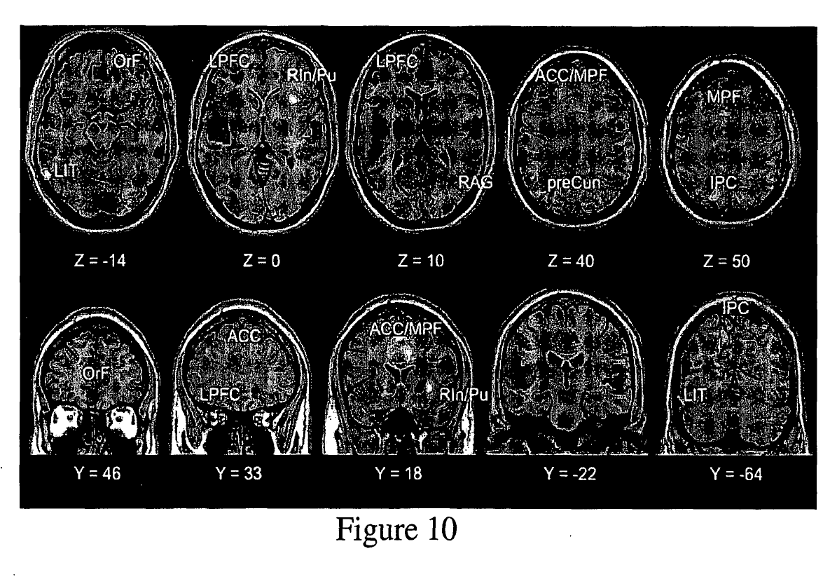

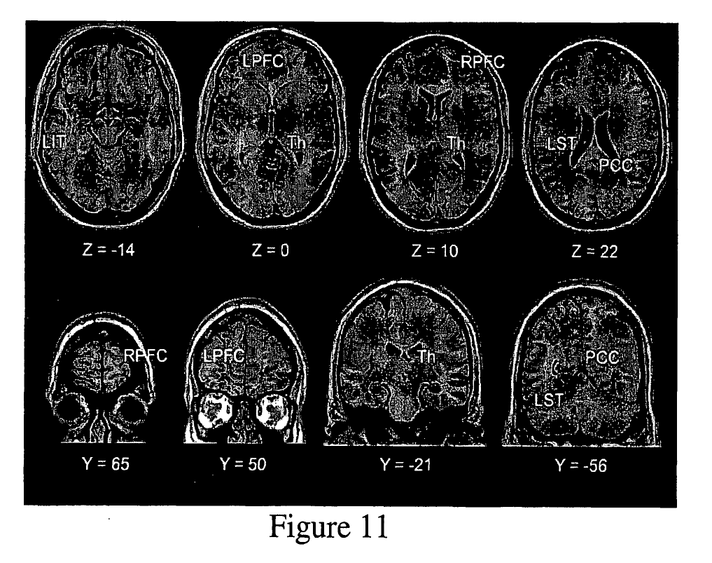

Within-subject comparison of CBF between the high- and low-stress tasks was carried out by using a random-effects model (see FIG. 10). Increased CBF was observed in the right insula/putamen, dorsomedial prefrontal cortex/ACC, precuneus/superior parietal gyrus, and left inferior temporal region. Suppressed CBF was observed in the ventrolateral left prefrontal cortex (LPFC) and orbitofrontal cortex (70% on the left side). In addition, there were bilateral deactivation clusters with reduced CBF during the high-stress task relative to the control condition, including pre- and postcentral gyri, insula, superior and middle temporal cortex, and right angular gyrus/inferior parietal cortex. The within-subject comparison of baseline CBF pre- and poststress tasks (baseline 2−baseline 1) revealed activation in the anterior RPFC, ventrolateral LPFC, thalamus, posterior cingulate cortex, and left inferior temporal cortex, whereas reduced CBF was observed only in the left superior temporal region (see FIG. 11).

### Example 2

### Gender Difference in Stress Response Revealed by Perfusion MRI

### Methods

Thirty-two healthy subjects (16 females and 16 males) were included in this study. The mean ages of the female and male group were 22.8±2.4 (SD) and 24.3±3.1 years (n.s.). The protocol consisted of 4 8-min perfusion fMRI scans: one low stress task (counting backward), one high stress task (serial subtraction of 13 under pressure), and two baseline scans before and after stress tasks. Self report of stress and anxiety level (1-9), heart rate (HR) as well as saliva samples were collected during the experiment (2). Perfusion fMRI data were analyzed using VoxBo and SPM2. After motion correction and spatial smoothing, label and control images were pair-wisely subtracted and converted to absolute perfusion image series. Voxel-wise analyses of the perfusion data were first conducted in each subject. Two contrasts were defined i.e., the perfusion difference during stress tasks (high−low stress) and the perfusion change at baseline (2nd−1st baseline). Individual contrast images were normalized into the MNI space, and linear regression analyses were carried out to obtain the activation pattern correlated with perceived stress, salivary cortisol level and gender. Activations were identified at P<0.005 (uncorrected) and cluster size >15 voxels.

### Results

### Behavioral and Physiological Stress Responses

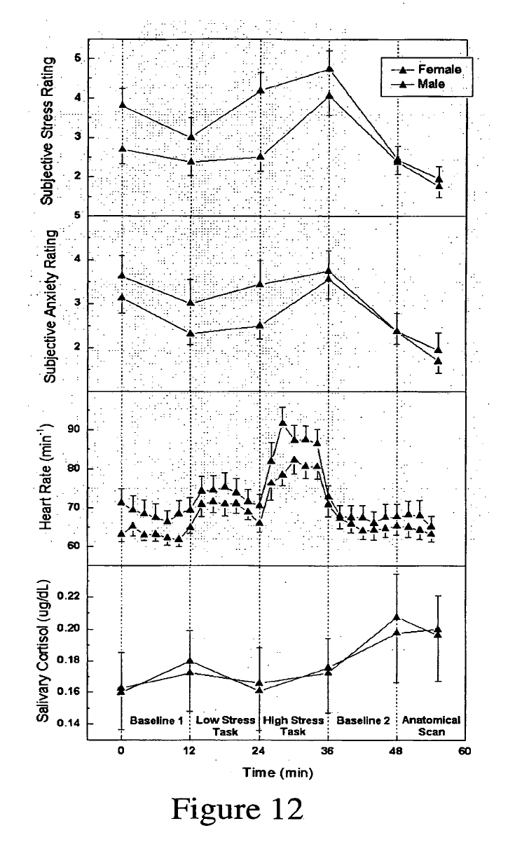

The measured behavioral and physiological data indicated that the experimental paradigm successfully elicited a mild to moderate level of psychological stress in both male and female subjects. The main effect of experimental condition was significant for perceived stress (F(5, 26)=17.47, P<0.001), perceived anxiety (F(5, 26)=19.55, P<0.001) and heart rate (F(4, 27)=41.76, P<0.001), which were immediately elevated in response to the stress tasks, as well as for salivary cortisol (F(5, 26)=3.22, P=0.021), which showed a delayed response to the high stress task (FIG. 12). The main effect of gender was not significant for perceived stress/anxiety, heart rate or salivary cortisol measures. However, the interaction of experimental condition and gender was significant for perceived stress (F(5, 26)=5.52, P=0.001). Post hoc analyses indicated that males reported a greater acute response in perceived stress from the low to high stress task (F(1, 30)=4.39, P=0.045) compared to females. This effect was not observed for perceived anxiety, although self ratings of stress and anxiety were correlated (R=0.76, P<0.001). Despite a higher level of task difficulty (F(1, 30)=7.20, P=0.012) and effort required (F(1, 30)=4.93, P=0.034) reported by females during the stress tasks, men and women performed equally well for the serial subtraction task. There was no significant difference between the two sexes in the recorded number of errors made (male: mean±SEM=5.7±1.0, female: 6.2±1.3, Z=0.17, P=0.87) and completed subtractions before committing an error (male: 19.2±3.4, female: 15.34±4.8, Z=1.35, P=0.18).

Neural Pathways Associated with Perceived Stress in Men and Women

The neural correlates of subjects' own experience of stress were probed using voxel-wise linear regression analyses of the perfusion fMRI data with perceived stress. First, acute stress responses during the performance of stress tasks were identified by correlating changes in regional CBF and perceived stress from the low to high stress task (High−Low stress task). Second, lasting stress effects after task completion were identified by correlating baseline CBF variations (Baseline 2−1) with changes in perceived stress from the low to high stress task.

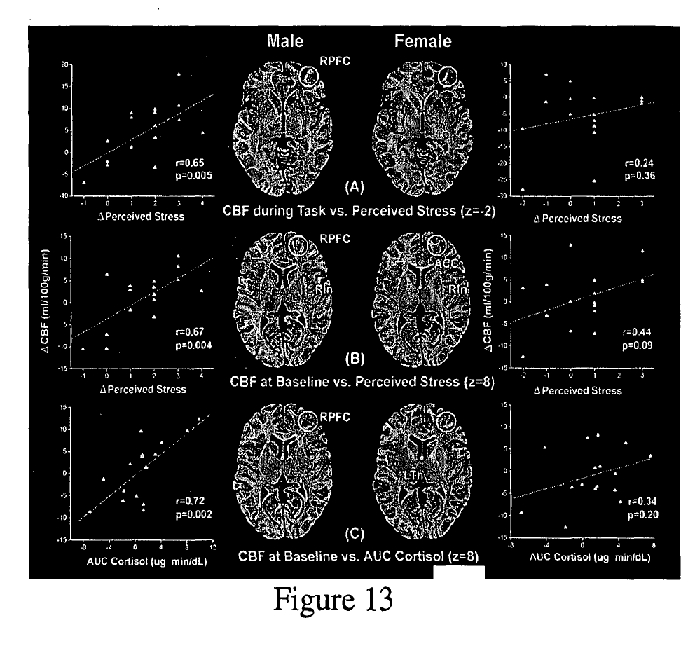

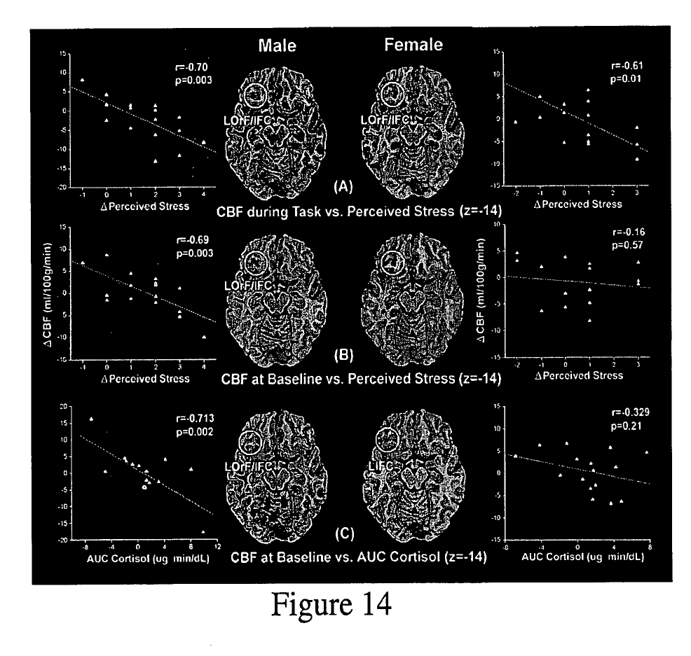

Performing the two regression analyses in each gender revealed that, in the male group, CBF in the RPFC was elevated both during the performance of stress tasks and at baseline after task completion in subjects experiencing stress. However, no significant correlation between RPFC activation and perceived stress was observed in the female group either during tasks or at baseline (FIGS. 13A&B). We further observed that, in the male group, CBF in the LOrF/inferior frontal cortex (IFC) was suppressed both during the performance of stress tasks and at baseline after task completion in subjects experiencing stress (FIGS. 14A&B). For females, the association of CBF reduction in LOrF/IFC and perceived stress was only significant during the performance of stress tasks (FIG. 14A). These results suggest that the stress response in men is primarily characterized by RPFC activation accompanied by LOrF/IFC inhibition, a robust response that persists beyond the stress task period. In contrast, women only showed transient suppression of the LOrF/IFC during the performance of stress tasks.

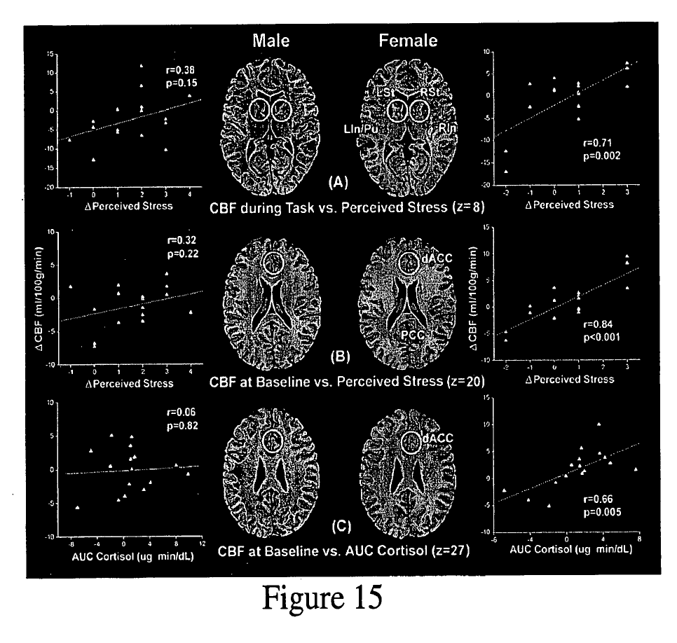

We then examined the limbic system along with closely interconnected brain regions including hippocampus, insula and cingulate cortex. During the performance of stress tasks, CBF increases in the left insula/putamen (LIn/Pu), right insula (RIn) and bilateral ventral striatum (LSt & RSt), including caudate and globus pallidus, were correlated with subjective stress ratings only in the female group. In contrast, the male group did not exhibit any stress related brain activation in the limbic regions during stress tasks (FIG. 15A). After completion of stress tasks, persistent activation in the ACC, posterior cingulate cortex (PCC) and RIn were associated with heightened stress level during tasks in the female group. In the male group, persistent CBF elevation was observed only in the RIn in stressed subjects (FIG. 13B). These results indicate that the female stress response is primarily associated with limbic activation of the ventral striatum, putamen and insula during stress tasks, and the ACC and PCC persisting beyond the task period.

Since women experienced increased cognitive demand relative to men during stress tasks, there exists the concern that our observation may reflect gender differences in performing arithmetic tasks rather than stress reactivity. We therefore repeated the above regression analyses while including subjective ratings of effort/difficulty as a covariate in conjunction with perceived stress. Behaviorally, subjective ratings of stress and effort/difficulty were not correlated (R=0.07, P=0.80). Including effort/difficulty as a covariate along with stress in regression analyses of CBF data did not affect the reported results on gender differences in brain activation associated with perceived stress.

### Example 3

### Neural Pathways Associated with Salivary Cortisol in Men and Women

The above results were based on regression of brain responses with subjective stress experience, which may differ between men and women. For instance, it has been reported that females may have a lower threshold for perceived stress compared to males since puberty. We therefore performed a third regression analysis to detect associations between baseline CBF variations (Baseline 2−1) and AUC measures of salivary cortisol—a physiological index of overall stress elevation caused by undergoing the experimental stress paradigm. Again, in the male subjects, we found that baseline CBF increase in the RPFC and CBF reduction in the LOrF/IFC were correlated with AUC measures of salivary cortisol (FIGS. 13C&14C). In contrast, significant cortisol related CBF increases were observed in the dorsal ACC (dACC) and left thalamus (LTh) only in the female but not the male group (FIGS. 13C&15C). Females also showed cortisol related CBF reduction in the left IFC (LIFC), but at a much weaker significance level compared to the LOrF/IFC suppression observed in males (FIG. 14C). These additional analyses relying on a physiological parameter—salivary cortisol—are consistent with our findings based on behavioral assessments of stress.

### Comparison of Average Stress Responses Between Men and Women

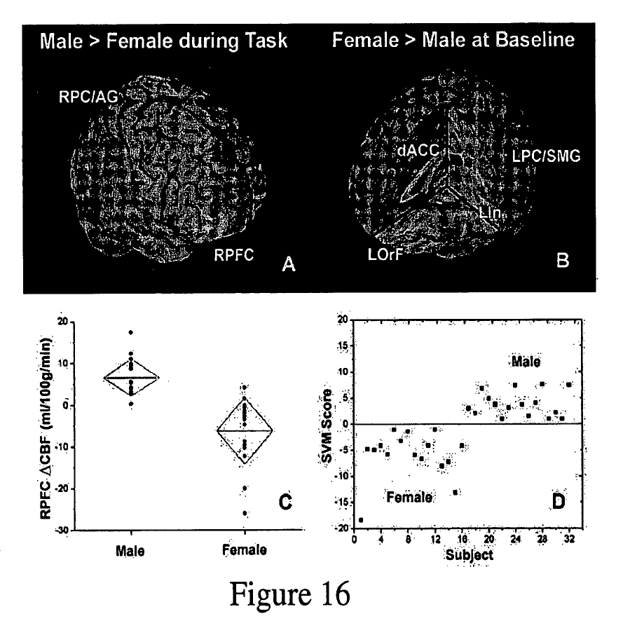

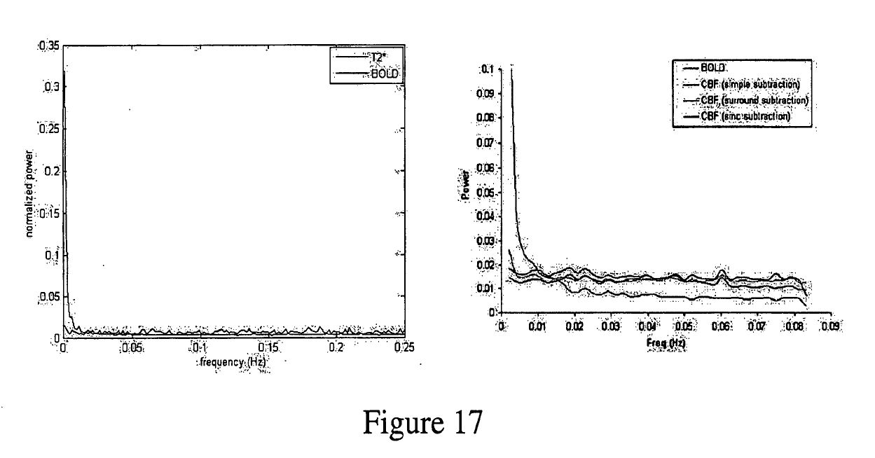

To address whether the average brain activation pattern under stress differs between men and women, we compared the mean acute (High−Low stress task) and persistent (Baseline 2−1) CBF responses to stress between the male and female group, using a regression analysis including gender as the independent variable (FIG. 16A) (i.e., unpaired t-test between male and female groups). During the stress tasks, men showed predominantly greater CBF augmentations than women in the right hemisphere including the RPFC and right parietal cortex/angular gyrus (RPC/AG), whereas women only showed greater activation in PCC compared to men. The greater acute RPFC activation in the male group was the most significant finding (peak Z=3.96). This activation survived the small volume corrected threshold (P=0.04) using the right frontal lobe as the search volume. When perceived stress was also included as a covariate along with gender in the regression analysis, this gender effect in the RPFC was still significant. Based on estimation of effect size using ANOVA, gender and perceived stress accounted for 51.5% (P<0.001) and 49.8% (P=0.015) of the total variance of RPFC CBF changes during stress tasks, respectively.

In contrast to the acute stress responses, during the post- vs. pre-stress baseline conditions, women showed much greater CBF elevations than men primarily in the left hemisphere, including the LOrF, left insula (LIn), dorsal ACC and left parietal cortex/supramarginal gyrus (LPC/SMG) (FIG. 16B), whereas men only showed greater activation in the right thalamus compared to women. Taken together, the group comparison results and the regression analyses carried out independently in the male and female groups suggest that the RPFC activation provides a unique biomarker of the acute stress response in men. In contrast, females show greater persistent activation of the dorsal ACC and Lin, and less suppression of the LOrF after task completion compared to men.

### Classification of Stress Responses in Men and Women

We further employed a support-vector-machine (SVM) based linear classification approach to differentiate the female and male stress response. As shown in FIG. 16C, the CBF changes in the RPFC from the low to high stress task provided a relatively clean separation between the male and female group, which yielded an accuracy of 93.8% (two errors in 32 subjects) for SVM classification based on just a single ROI of the RPFC. We then sequentially included corresponding CBF changes in stress-related brain regions demonstrating gender differences into the SVM classification, including the LOrF, dorsal ACC and LIn. We were able to achieve a perfect (100%) separation of the male and female group when all 4 ROIs were included (FIG. 16D). The RPFC was the most important factor in the SVM classifier, with a weighting factor of 51.5%.

### Example 4

### Temporal Stability of Perfusion MRI and absolute T2 mapping MRI

### Methods

One healthy subject was scanned on a 3T Siemens Trio MRI system using standard BOLD fMRI and absolute T2 mapping MRI. BOLD fMRI used gradient-echo echo-planar imaging (EPI) sequence with imaging parameters of TR/TE=2000/30 ms, 25 slices 4 mm thickness, flip-angle=80. Absolute T2 mapping used double-echo gradient echo EPI sequences with imaging parameters of TR=2 s, TE1=19 ms, TE2=49 ms, 25 slices 4 mm thickness, flip-angle=80. 5 min of resting state data were acquired from the subject using BOLD fMRI and absolute T2 mapping MRI respectively. T2* values were calculated using an exponential decay model. Power spectra of both BOLD and T2* image series were calculated in each pixel, followed by averaging across the whole brain to generate the global mean power spectra.

Ten healthy subjects were scanned on a 4T GE MRI system using a pulsed ASL technique, imaging parameters were: TR/TE=3000/20 ms, 8 slices 10 mm thickness, flip-angle=90, label time=700 ms, delay time=800 ms, gradient-echo EPI. 10 min resting state data were acquired. BOLD and CBF image series were generated by pair-wise summation and subtraction (simple, surround and sinc respectively) of label and control images. The mean power spectra of BOLD and ASL data were generated for each image series.

### Results

As shown in FIG. 17, while the power spectra of BOLD fMRI data consistently showed increased power at low temporal frequencies, both T2* and ASL image series show even distribution of power across the spectrum. This results means that T2* and ASL image series are very stable in time and can be used to visualized slow changes in brain function and sustained behavioral states such as stress.

1. A method for quantifying subject's reactivity to psychological stress comprising: establishing a cerebral blood flow (CBF) perfusion baseline, or blood oxygenation baseline of the subject, or their combination for the subject, using scanning with arterial spin labeling (ASL) perfusion magnetic resonance imaging (MRI) or absolute T2 mapping MRI; inducing stress in the subject, while the subject is undergoing MRI scanning with arterial spin labeling (ASL) perfusion magnetic resonance imaging (MRI) or absolute T2 mapping MRI; determining changes in the cerebral blood flow (CBF), or blood oxygenation in a brain region associated with stress responses; and comparing the captured changes in a blood flow, or a blood oxygenation pattern with changes in a blood flow, or a blood oxygenation pattern in a reference database, wherein the reference database indicates reactivity to psychological stress of a predetermined individual or pool of individuals.

2. The method of claim 1, wherein the brain regions associated with stress are the right prefrontal cortex (RPFC), left prefrontal cortex, left orbitofrontal cortex, anterior cingulate cortex (ACC), insula, puteman, amygdala, striatum, nucleus accumbens (NA), hippocampus or a combination thereof.

3. The method of claim 1, wherein said step of inducing stress in the subject comprises making the subject perform psychomotor vigilance task (PVT); probed recall memory (PRM); visual memory task (VMT); synthetic workload task (SYNW); Stroop tasks, mirror tracing, solving puzzles, making an anagram, meter reading task (MRT); logical reasoning task (LRT); Haylings sentence completion (HSC), arithmetical tasks; public speaking, interview or verbal interactions, emotion induction paradigms such as imagining or recalling dysphoric or stressful experiences, watching disturbing or fearful video or pictures, listening to depressing audio, exposure to noise, or a combination thereof, whereby time and performance pressure, negative psychosocial feedback and their combinations are provided to the subjects to elicit robust stress responses.

4. (canceled)

5. (canceled)

6. The method of claim 1, further comprising the step of reestablishing a perfusion baseline, a blood oxygenation baseline or a combination thereof of the subject's brain using ASL perfusion MRI scanning or absolute T2 mapping MRI, following the step of inducing stress in the subject.

7. The method of claim 1, further comprising the steps of collecting additional data between the steps of establishing a baseline and the step of inducing stress; between the step of inducing stress and the step of imaging cerebral blood flow (CBF) changes, blood oxygenation changes, or a combination thereof; and after the step of imaging cerebral blood flow (CBF) changes, blood oxygenation changes or a combination thereof.

8. The method of claim 4, wherein the additional data is saliva samples, blood sample, heart rate, blood pressure, skin conductance and subjective ratings of stress, anxiety, fatigue, depression or a combination thereof.

9. The method of claim 1, wherein the reference database comprises the captured image of cerebral blood flow (CBF) changes, blood oxygenation changes, or a combination thereof taken from a brain regions associated with stress of a predetermined subject or pool of subjects.

10. The method of claim 7, wherein the predetermined subject or pool of subjects is selected from top executives, elite athletes, performers, astronauts, air traffic controllers, combat soldiers, political leaders, or a combination thereof.

11. The method of claim 7, wherein the predetermined subject or pool of subjects is selected from paranoid schizophrenics, drug addicts, depressives, phobics, subjects afflicted with obesity, hypertension, diabetes, obsessive compulsive disorder, post-traumatic stress syndrome, or a combination thereof.

12. The method of claim 1, further comprising the step of identifying candidates with brain activation to stress matching those of the predetermined individual or pool of individuals.

13. The method of claim 1, further comprising the step of identifying candidates with brain activation to stress dissimilar to those of the predetermined individual or pool of individuals.

14. (canceled)

15. A method of screening candidates for a high-stress position comprising the steps of: establishing a cerebral blood flow (CBF) perfusion baseline, blood oxygenation baseline, or a combination thereof for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF) perfusion baseline, blood oxygenation baseline, or a combination thereof in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern, or their combination with changes in blood flow pattern, blood oxygenation pattern, or their combination in a reference database, wherein the reference database indicates reactivity to psychological stress of a predetermined individual or pool of individuals proven as appropriate for the high-stress position sought to be screened for.

16. The method of claim 12, wherein the brain regions associated with stress are the right prefrontal cortex (RPFC), left prefrontal cortex, left orbitofrontal cortex, the anterior cingulate cortex (ACC), insula, puteman, amygdala, striatum, nucleus accumbens (NA), hippocampus, or a combination thereof.

17. The method of claim 12, wherein the step of inducing stress comprises inducing stress typical of the position for which the screening is sought.

18. The method of claim 12, further comprising the steps of collecting additional data between the steps of establishing a baseline and the step of inducing stress; between the step of inducing stress and the step of imaging cerebral blood flow (CBF) changes, blood oxygenation changes or their combination; and after the step of imaging cerebral blood flow (CBF) changes, blood oxygenation changes, or their combination.

19. (canceled)

20. The method of claim 15, wherein the additional data is saliva samples, blood samples, heart rate, blood pressure, skin conductance and subjective ratings of stress, anxiety, fatigue, depression or a combination thereof.

21. A method of diagnosing a mental disorder associated with a subject's susceptibility to psychological stress comprising the steps of establishing a cerebral blood flow (CBF) perfusion baseline, blood oxygenation baseline or their combination for the subject, using scanning with arterial spin labeling (ASL) perfusion magnetic resonance imaging (MRI) or absolute T2 mapping MRI; inducing stress in the subject, while the subject is undergoing scanning with arterial spin labeling (ASL) perfusion magnetic resonance imaging (MRI) or absolute T2 mapping MRI; capturing changes in the cerebral blood flow (CBF), blood oxygenation, or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern, or their combination with changes in blood flow pattern, blood oxygenation pattern, or their combination in a reference database, wherein the reference database indicates reactivity to psychological stress of a predetermined individual or pool of individuals correctly diagnosed with said mental disorder sought to be diagnosed.

22. The method of claim 17, wherein the brain regions associated with stress are the right prefrontal cortex (RPFC), left prefrontal cortex, left orbitofrontal cortex, the anterior cingulate cortex (ACC), insula, puteman, amygdala, striatum, nucleus accumbens (NA), hippocampus, or a combination thereof.

23. The method of claim 17, wherein the step of inducing stress comprises inducing stress typical of the stress triggering the mental disorder sought to be diagnosed.

24. The method of claim 17, further comprising the steps of collecting additional data between the steps of establishing a baseline and the step of inducing stress; between the step of inducing stress and the step of imaging cerebral blood flow (CBF) changes, blood oxygenation changes or their combination; and after the step of imaging cerebral blood flow (CBF) changes, blood oxygenation changes, or their combination.

25. (canceled)

26. The method of claim 20, wherein the additional data is saliva samples, blood samples, heart rate, blood pressure, skin conductance and subjective ratings of stress, anxiety, fatigue, depression or a combination thereof.

27. The method of claim 17, wherein the mental disorder is post-traumatic stress disorder (PTSD), anxiety disorder, social phobia, depression and obsessive compulsive disorder (OCD).

28. A library of images of cerebral blood flow changes, blood oxygenation changes or their combination in brain regions associated with stress response, wherein the images are captured in response to psychological stress, using scanning with arterial spin labeling (ASL) perfusion magnetic resonance imaging (MRI) or absolute T2 mapping MRI, taken from a predetermined subject or pool of subjects.

29. The library of claim 23, wherein the brain regions associated with stress are the right prefrontal cortex (RPFC), left prefrontal cortex, left orbitofrontal cortex, the anterior cingulate cortex (ACC), insula, puteman, amygdala, striatum, nucleus accumbens (NA), hippocampus, or a combination thereof.

30. The library of claim 23, wherein the predetermined subject or pool of subjects is selected from top executives, elite athletes, performers, astronauts, air traffic controllers, combat soldiers, political leaders or a combination thereof.

31. The library of claim 23, wherein the predetermined subject or pool of subjects is selected from paranoid schizophrenics, drug addicts, manic depressives, phobics, subjects afflicted with obesity, hypertension, diabetes, obsessive compulsive disorder, post-traumatic stress syndrome, or a combination thereof.

32. A machine readable media comprising the library of claim 23.

33. The machine readable media of claim 23, wherein the images are searchable by a predetermined criterion.

34. The machine readable media of claim 28, wherein the predetermined criterion is gender, age similarity of the stress-induced brain activation pattern of cerebral blood flow, blood oxygenation or their combination, detected in one given subject, the predetermined individual or pool of individuals related to the purpose of screening or selection.

35. A method of testing a candidate drug as an psychotherapeutic drug, or optimizing a level of a psychotherapeutic drug comprising the step of: deviding a cohort of subjects into two groups, administering to one group a placebo and to the other group the candidate drug or the drug sought to be optimized; establishing a cerebral blood flow (CBF) perfusion baseline, blood oxygenation baseline or their combination for both groups, using scanning with arterial spin labeling (ASL) perfusion magnetic resonance imaging (MRI) or absolute T2 mapping MRI; inducing stress in both groups, while individuals in the groups are undergoing scanning with arterial spin labeling (ASL) perfusion magnetic resonance imaging (M) or absolute T2 mapping MRI; capturing changes in the cerebral blood flow (CBF), blood oxygenation or their combination in brain regions associated with stress responses, wherein the changes are captured during the scanning with arterial spin labeling (ASL) perfusion magnetic resonance imaging (MRI) or absolute T2 mapping MRI; and comparing the captured changes in blood flow pattern, blood oxygenation pattern, or their combination between the individuals in the group that received placebo, with the individuals in the group that received the candidate drug or the drug sought to be optimized, wherein blood flow pattern, blood oxygenation pattern, or a combination thereof in the group which received the candidate drug or the drug sought to be optimized, which yields cerebral blood flow pattern, blood oxygenation pattern, or a combination thereof, which resembles the baseline cerebral blood flow pattern, blood oxygenation pattern, or a combination thereof, which is closer than that of the cerebral blood flow pattern, blood oxygenation pattern of a combination thereof, of the group that received placebo or the drug sought to be optimized, indicate the candidate drug is a psychotherapeutic drug or a drug with an optimized level.

36. The method of claim 30, wherein the brain regions associated with stress are the right prefrontal cortex (RPFC), left prefrontal cortex, left orbitofrontal cortex, the anterior cingulate cortex (ACC), insula, puteman, amygdala, striatum, nucleus accumbens (NA), hippocampus, or a combination thereof.

37. The method of claim 30, wherein the step of inducing stress comprises inducing stress typical of the stress triggering the psychiatric condition sought to be targeted, or for which the drug is sought to be optimized.

38. The method of claim 32, wherein the psychiatric condition is depression, dementia, night terrors, drug addiction, auto-immune diseases, obsessive-compulsive disorder, panic attacks, post-traumatic stress syndrome (PTSD), social phobia or anxiety.

39. The method of claim 30, further comprising the steps of collecting additional data between the steps of establishing a baseline and the step of inducing stress; between the step of inducing stress and the step of imaging cerebral blood flow (CBF) changes, blood oxygenation changes or their combination; and after the step of imaging cerebral blood flow (CBF) changes, blood oxygenation changes, or their combination.

40. The method of claim 34, wherein the additional data is saliva samples, blood samples, heart rate, blood pressure, skin conductance and subjective ratings of stress, anxiety, fatigue, depression or a combination thereof.

41. (canceled)

42. The method of claim 35, wherein the psychotherapeutic drug sought to be optimized is tranquilizer, beta-blocker, sleeping pill, or antidepressant.

43. (canceled)

44. (canceled)

45. (canceled)

46. (canceled)

47. (canceled)
